SEQUENCE LISTING (133 sequences)

SEQ ID NO: 1 - DNA, 650 bp, H. sapiens
acagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccaaaaaaaaaaaaaaaaaaaaaaa

SEQ ID NO: 2 - DNA, 8054 bp, H. sapiens
ttgttgacccatggatccatcaagtgcaaacattttctaatgcactatatttaagcctgtgcagctagatgtcattcaacatgaaatacattattacaacttgcatctgtctaaaatcttgcatctaaaatgagagacaaaaaatctataaaaatggaaaacatgcatagaaatatgtgagggaggaaaaaattacccccaagaatgttagtgcacgcagtcacacagggagaagactatttttgttttgttttgattgttttgttttgttttggttgttttgttttggtgacctaactggtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttggcaggatggcttctcatcgtctgctcctcctctgccttgctggactggtatttgtgtctgaggctggccctacggtgagtgtttctgtgacatcccattcctacatttaagattcacgctaaatgaagtagaagtgactccttccagctttgccaaccagcttttattactagggcaagggtacccagcatctatttttaatataattaattcaaacttcaaaaagaatgaagttccactgagcttactgagctgggacttgaactctgagcattctacctcattgctttggtgcattaggtttgtaatatctggtacctctgtttcctcagatagatgatagaaataaagatatgatattaaggaagctgttaatactgaattttcagaaaagtatccctccataaaatgtatttgggggacaaactgcaggagattatattctggccctatagttattcaaaacgtatttattgattaatctttaaaaggcttagtgaacaatattctagtcagatatctaattcttaaatcctctagaagaattaactaatactataaaatgggtctggatgtagttctgacattattttataacaactggtaagagggagtgactatagcaacaactaaaatgatctcaggaaaacctgtttggccctatgtatggtacattacatcttttcagtaattccactcaaatggagacttttaacaaagcaactgttctcaggggacctattttctcccttaaaattcattatacacatccctggttgatagcagtgtgtctggaggcagaaaccattcttgctttggaaacaattacgtctgtgttatactgagtagggaagctcattaattgtcgacacttacgttcctgataatgggatcagtgtgtaattcttgtttcgctccagatttctaataccacaaagaataaatcctttcactctgatcaattttgttaacttctcacgtgtcttctctacacccagggcaccggtgaatccaagtgtcctctgatggtcaaagttctagatgctgtccgaggcagtcctgccatcaatgtggccgtgcatgtgttcagaaaggctgctgatgacacctgggagccatttgcctctgggtaagttgccaaagaaccctcccacaggacttggttttatcttcccgtttgcccctcacttggtagagagaggctcacatcatctgctaaagaatttacaagtagattgaaaaacgtaggcagaggtcaagtatgccctctgaaggatgccctctttttgttttgcttagctaggaagtgaccaggaacctgagcatcatttaggggcagacagtagagaaaagaaggaatcagaactcctctcctctagctgtggtttgcaacccttttgggtcacagaacactttatgtaggtgatgaaaagtaaacattctatgcccagaaaaaatgcacagatacacacacatacaaaatcatatatgtgattttaggagtttcacagattccctggtgtccctgggtaacaccaaagctaagtgtccttgtcttagaattttaggaaaaggtataatgtgtattaacccattaacaaaaggaaaggaattcagaaatattattaaccaggcatctgtctgtagttaatatggatcacccaaaacccaaggcttttgcctaatgaacactttggggcacctactgtgtgcaaggctgggggctgtcaagctcagttaaaaaaaaaaagatagaagagatggatccatgaggcaaagtacagccccaggctaatcccacgatcacccgacttcatgtccaagagtggcttctcaccttcattagccagttcacaattttcatggagtttttctacctgcactagcaaaaacttcaaggaaaatacatattaataaatctaagcaaagtgaccagaagacagagcaatcaggagaccctttgcatccagcagaagaggaactgctaagtatttacatctccacagagaagaatttctgttgggttttaattgaaccccaagaaccacatgattcttcaaccattattgggaagatcattttcttaggtctggttttaactggctttttatttgggaattcatttatgtttatataaaatgccaagcataacatgaaaagtggttacaggactattctaagggagagacagaatggacaccaaaaatattccaatgttcttgtgaatcttttccttgcaccaggacaaaaaaaaaaagaagtgaaaagaagaaaggaggaggggcataatcagagtcagtaaagacaactgctatttttatctatcgtagctgttgcagtcaaatgggaagcaatttccaacattcaactatggagctggtacttacatggaaatagaagttgcctagtgtttgttgctggcaaagagttatcagagaggttaaatatataaaagggaaaagagtcagatacaggttcttcttcctactttaggttttccactgtgtgtgcaaatgatactccctggtggtgtgcagatgcctcaaagctatcctcacaccacaagggagaggagcgagatcctgctgtcctggagaagtgcagagttagaacagctgtggccacttgcatccaatcatcaatcttgaatcacagggactctttcttaagtaaacattatacctggccgggcacggtggctcacgcctgtaatcccagcactttgggatgccaaagtgggcatatcatctgaggtcaggagttcaagaccagcctggccaacatggcaaaactccgtctttatgaaaaatacaaaaattagccaggcatggtggcaggcgcctgtaatcccagctaattgggaggctgaggctggagaatcccttgaatctaggaggcagaggttgcagtgagctgagatcgtgccattgcactccagcctgggtgacaagagtaaaactctgtctcaaaaaaaaaaaattatacctacattctcttcttatcagagaaaaaaatctacagtgagcttttcaaaaagtttttacaaactttttgccatttaatttcagttaggagttttccctacttctgacttagttgaggggaaatgttcataacatgtttataacatgtttatgtgtgttagttggtgggggtgtattactttgccatgccatttgtttcctccatgcgtaacttaatccagactttcacaccttataggaaaaccagtgagtctggagagctgcatgggctcacaactgaggaggaatttgtagaagggatatacaaagtggaaatagacaccaaatcttactggaaggcacttggcatctccccattccatgagcatgcagaggtgagtatacagaccttcgagggttgttttggttttggtttttgcttttggcattccaggaaatgcacagttttactcagtgtaccacagaaatgtcctaaggaaggtgatgaatgaccaaaggttccctttcctattatacaagaaaaaattcacaacactctgagaagcaaatttctttttgactttgatgaaaatccacttagtaacatgacttgaacttacatgaaactactcatagtctattcattccactttatatgaatattgatgtatctgctgttgaaataatagtttatgaggcagccctccagaccccacgtagagtgtatgtaacaagagatgcaccattttatttctcgaaaacccgtaacattcttcattccaaaacacatctggcttctcggaggtctggacaagtgattcttggcaacacatacctatagagacaataaaatcaaagtaataatggcaacacaatagataacatttaccaagcatacaccatgtggcagacacaattataagtgttttccatatttaacctacttaatcctcaggaataagccactgaggtcagtcctattattatccccatcttatagatgaagaaaatgaggcaccaggaagtcaaataacttgtcaaaggtcacaagactaggaaatacacaagtagaaatgtttacaattaaggcccaggctgggtttgccctcagttctgctatgcctcgcattatgccccaggaaactttttcccttgtgaaagccaagcttaaaaaaagaaaagccacatttgtaacgtgctctgttcccctgcctatggtgaggatcttcaaacagttatacatggacccagtccccctgccttctccttaatttcttaagtcatttgaaacagatggctgtcatggaaatagaatccagacatgttggtcagagttaaagatcaactaattccatcaaaaatagctcggcatgaaagggaactattctctggcttagtcatggatgagactttcaattgctataaagtggttcctttattagacaatgttaccagggaaacaacaggggtttgtttgacttctggggcccacaagtcaacaagagagccccatctaccaaggagcatgtccctgactacccctcagccagcagcaagacatggaccccagtcagggcaggagcagggtttcggcggcgcccagcacaagacattgcccctagagtctcagcccctaccctcgagtaatagatctgcctacctgagactgttgtttgcccaagagctgggtctcagcctgatgggaaccatataaaaaggttcactgacatactgcccacatgttgttctctttcattagatcttagcttccttgtctgctcttcattcttgcagtattcattcaacaaacattaaaaaaaaaaaaaagcattctatgtgtggaacactctgctagatgctgtggatttagaaatgaaaatacatcccgacccttggaatggaagggaaaggactgaagtaagacagattaagcaggaccgtcagcccagcttgaagcccagataaatacggagaacaagagagagcgagtagtgagagatgagtcccaatgcctcactttggtgacgggtgcgtggtgggcttcatgcagcttcttctgataaatgcctccttcagaactggtcaactctaccttggccagtgacccaggtggtcatagtagatttaccaagggaaaatggaaacttttattaggagctcttaggcctcttcacttcatggatttttttttcctttttttttgagatggagttttgccctgtcacccaggctggaatgcagtggtgcaatctcagctcactgcaacctccgcctcccaggttcaagcaattctcctgcctcagcctcccgagtagctgggactacaggtgtgcgccaccacaccaggctaatttttgtattttttgtaaagacaggttttcaccacgttggccaggctggtctgaactccagacctcaggtgattcacctgtctcagcctcccaaagtgctgggattacaggtgtgagccaccgtgcccggctacttcatggatttttgattacagattatgcctcttacaatttttaagaagaatcaagtgggctgaaggtcaatgtcaccataagacaaaagacatttttattagttgattctagggaattggccttaaggggagccctttcttcctaagagattcttaggtgattctcacttcctcttgccccagtattatttttgtttttggtatggctcactcagatccttttttcctcctatccctaagtaatccgggtttctttttcccatatttagaacaaaatgtatttatgcagagtgtgtccaaacctcaacccaaggcctgtatacaaaataaatcaaattaaacacatctttactgtcttctacctctttcctgacctcaatatatcccaacttgcctcactctgagaaccaaggctgtcccagcacctgagtcgcagatattctactgatttgacagaactgtgtgactatctggaacagcattttgatccacaatttgcccagttacaaagcttaaatgagctctagtgcatgcatatatatttcaaaattccaccatgatcttccacactctgtattgtaaatagagccctgtaatgcttttacttcgtatttcattgcttgttatacataaaaatatacttttcttcttcatgttagaaaatgcaaagaataggagggtgggggaatctctgggcttggagacaggagacttgccttcctactatggttccatcagaatgtagactgggacaatacaataattcaagtctggtttgctcatctgtaaattgggaagaatgtttccagctccagaatgctaaatctctaagtctgtggttggcagccactattgcagcagctcttcaatgactcaatgcagttttgcattctccctaccttttttttctaaaaccaataaaatagatacagcctttaggctttctgggatttcccttagtcaagctagggtcatcctgactttcggcgtgaatttgcaaaacaagacctgactctgtactcctgctctaaggactgtgcatggttccaaaggcttagcttgccagcatatttgagctttttccttctgttcaaactgttccaaaatataaaagaataaaattaattaagttggcactggacttccggtggtcagtcatgtgtgtcatctgtcacgtttttcgggctctggtggaaatggatctgtctgtcttctctcataggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtaaatgagagagccttattttcctgtaaccttcagcaaatagcaaaagacacattccaagggcccacttctttactgtgggcatttctttttttttctttttttcttttttccttttttgagacaaagtctcactctgttgcccaggctagaatgcagtggtgtaatctcagctcactgcaacctctgcttcctgggttcaagcgattctcctgcctcagcctcccaagtaactgggattacaggcgcatgccaccacgcctagctcatttttgtatttttagtagagatgggattttgccatgttggctaggctggtctacgaactcctgacctcaggtgatccacctgcctcagcctcccaaagtgctgggattacaggcatgagccactacacccgg

SEQ ID NO: 3 - DNA, 20 bp, Artificial Sequence
  Primer
ccgaggcagtcctgccatca

SEQ ID NO: 4 - DNA, 20 bp, Artificial Sequence
  Primer
gctcccaggtgtcatcagca

SEQ ID NO: 5 - DNA, 26 bp, Artificial Sequence
  Probe
tgtggccgtgcatgtgttcagaaagg

SEQ ID NO: 6 - DNA, 19 bp, Artificial Sequence
  Primer
ggcccgagaagacctcctt

SEQ ID NO: 7 - DNA, 21 bp, Artificial Sequence
  Primer
tcaatggtgcctctggagatt

SEQ ID NO: 8 - DNA, 22 bp, Artificial Sequence
  Probe
ccaggctttgggcatcaccacg

SEQ ID NO: 9 - DNA, 20 bp, Artificial Sequence
  Synthetic oligonucleotide
gtgcgcgcgagcccgaaatc

SEQ ID NO: 10-11 - skipped (2 entries)

SEQ ID NO: 12 - DNA, 20 bp, Artificial Sequence
  Synthetic oligonucleotide
aaacactcaccgtagggcca

SEQ ID NO: 13 - DNA, 20 bp, Artificial Sequence
  Synthetic oligonucleotide
caccggtgccctgggtgtag

SEQ ID NO: 14 - DNA, 20 bp, Artificial Sequence
  Synthetic oligonucleotide
tgagcctctctctaccaagt

SEQ ID NO: 15 - DNA, 20 bp, Artificial Sequence
  Synthetic oligonucleotide
gtatactcacctctgcatgc

SEQ ID NO: 16 - DNA, 20 bp, Artificial Sequence
  Synthetic oligonucleotide
ttctcagagtgttgtgaatt

SEQ ID NO: 17 - DNA, 20 bp, Artificial Sequence
  Synthetic oligonucleotide
actctgcataaatacatttt

SEQ ID NO: 18 - DNA, 20 bp, Artificial Sequence
  Synthetic oligonucleotide
tcttgttttgcaaattcacg

SEQ ID NO: 19 - DNA, 20 bp, Artificial Sequence
  Synthetic oligonucleotide
tgaataccacctatgagaga

SEQ ID NO: 20 - DNA, 20 bp, Artificial Sequence
  Synthetic oligonucleotide
ctgccaagaatgagtggact

SEQ ID NO: 21 - DNA, 20 bp, Artificial Sequence
  Synthetic oligonucleotide
tgagaagccatcctgccaag

SEQ ID NO: 22 - DNA, 20 bp, Artificial Sequence
  Synthetic oligonucleotide
cagacgatgagaagccatcc

SEQ ID NO: 23 - DNA, 20 bp, Artificial Sequence
  Synthetic oligonucleotide
aggagcagacgatgagaagc

SEQ ID NO: 24 - DNA, 20 bp, Artificial Sequence
  Synthetic oligonucleotide
acacaaataccagtccagca

SEQ ID NO: 25 - DNA, 20 bp, Artificial Sequence
  Synthetic oligonucleotide
gacacaaataccagtccagc

SEQ ID NO: 26 - DNA, 20 bp, Artificial Sequence
  Synthetic oligonucleotide
gcctcagacacaaataccag

SEQ ID NO: 27 - DNA, 20 bp, Artificial Sequence
  Synthetic oligonucleotide
gtagggccagcctcagacac

SEQ ID NO: 28 - DNA, 20 bp, Artificial Sequence
  Synthetic oligonucleotide
caccggtgcccgtagggcca

SEQ ID NO: 29 - DNA, 20 bp, Artificial Sequence
  Synthetic oligonucleotide
ggattcaccggtgcccgtag

SEQ ID NO: 30 - DNA, 20 bp, Artificial Sequence
  Synthetic oligonucleotide
aggacacttggattcaccgg

SEQ ID NO: 31 - DNA, 20 bp, Artificial Sequence
  Synthetic oligonucleotide
atcagaggacacttggattc

SEQ ID NO: 32 - DNA, 20 bp, Artificial Sequence
  Synthetic oligonucleotide
tgaccatcagaggacacttg

SEQ ID NO: 33 - DNA, 20 bp, Artificial Sequence
  Synthetic oligonucleotide
actttgaccatcagaggaca

SEQ ID NO: 34 - DNA, 20 bp, Artificial Sequence
  Synthetic oligonucleotide
acagcatctagaactttgac

SEQ ID NO: 35 - DNA, 20 bp, Artificial Sequence
  Synthetic oligonucleotide
gcctcggacagcatctagaa

SEQ ID NO: 36 - DNA, 20 bp, Artificial Sequence
  Synthetic oligonucleotide
tgatggcaggactgcctcgg

SEQ ID NO: 37 - DNA, 20 bp, Artificial Sequence
  Synthetic oligonucleotide
ttctgaacacatgcacggcc

SEQ ID NO: 38 - DNA, 20 bp, Artificial Sequence
  Synthetic oligonucleotide
tgtcatcagcagcctttctg

SEQ ID NO: 39 - DNA, 20 bp, Artificial Sequence
  Synthetic oligonucleotide
atggctcccaggtgtcatca

SEQ ID NO: 40 - DNA, 20 bp, Artificial Sequence
  Synthetic oligonucleotide
aggcaaatggctcccaggtg

SEQ ID NO: 41 - DNA, 20 bp, Artificial Sequence
  Synthetic oligonucleotide
ttcccagaggcaaatggctc

SEQ ID NO: 42 - DNA, 20 bp, Artificial Sequence
  Synthetic oligonucleotide
actggttttcccagaggcaa

SEQ ID NO: 43 - DNA, 20 bp, Artificial Sequence
  Synthetic oligonucleotide
gactcactggttttcccaga

SEQ ID NO: 44 - DNA, 20 bp, Artificial Sequence
  Synthetic oligonucleotide
cagctctccagactcactgg

SEQ ID NO: 45 - DNA, 20 bp, Artificial Sequence
  Synthetic oligonucleotide
gcccatgcagctctccagac

SEQ ID NO: 46 - DNA, 20 bp, Artificial Sequence
  Synthetic oligonucleotide
tgtgagcccatgcagctctc

SEQ ID NO: 47 - DNA, 20 bp, Artificial Sequence
  Synthetic oligonucleotide
ctcagttgtgagcccatgca

SEQ ID NO: 48 - DNA, 20 bp, Artificial Sequence
  Synthetic oligonucleotide
attcctcctcagttgtgagc

SEQ ID NO: 49 - DNA, 20 bp, Artificial Sequence
  Synthetic oligonucleotide
tctacaaattcctcctcagt

SEQ ID NO: 50 - DNA, 20 bp, Artificial Sequence
  Synthetic oligonucleotide
ctttgtatatcccttctaca

SEQ ID NO: 51 - DNA, 20 bp, Artificial Sequence
  Synthetic oligonucleotide
agatttggtgtctatttcca

SEQ ID NO: 52 - DNA, 20 bp, Artificial Sequence
  Synthetic oligonucleotide
caagtgccttccagtaagat

SEQ ID NO: 53 - DNA, 20 bp, Artificial Sequence
  Synthetic oligonucleotide
gggagatgccaagtgccttc

SEQ ID NO: 54 - DNA, 20 bp, Artificial Sequence
  Synthetic oligonucleotide
tctgcatgctcatggaatgg

SEQ ID NO: 55 - DNA, 20 bp, Artificial Sequence
  Synthetic oligonucleotide
tgaataccacctctgcatgc

SEQ ID NO: 56 - DNA, 20 bp, Artificial Sequence
  Synthetic oligonucleotide
ttggctgtgaataccacctc

SEQ ID NO: 57 - DNA, 20 bp, Artificial Sequence
  Synthetic oligonucleotide
ccggagtcgttggctgtgaa

SEQ ID NO: 58 - DNA, 20 bp, Artificial Sequence
  Synthetic oligonucleotide
tcagcagggcggcaatggtg

SEQ ID NO: 59 - DNA, 20 bp, Artificial Sequence
  Synthetic oligonucleotide
ccgtggtggaataggagtag

SEQ ID NO: 60 - DNA, 20 bp, Artificial Sequence
  Synthetic oligonucleotide
agccgtggtggaataggagt

SEQ ID NO: 61 - DNA, 20 bp, Artificial Sequence
  Synthetic oligonucleotide
cgacagccgtggtggaatag

SEQ ID NO: 62 - DNA, 20 bp, Artificial Sequence
  Synthetic oligonucleotide
ttggtgacgacagccgtggt

SEQ ID NO: 63 - DNA, 20 bp, Artificial Sequence
  Synthetic oligonucleotide
gattggtgacgacagccgtg

SEQ ID NO: 64 - DNA, 20 bp, Artificial Sequence
  Synthetic oligonucleotide
gggattggtgacgacagccg

SEQ ID NO: 65 - DNA, 20 bp, Artificial Sequence
  Synthetic oligonucleotide
tgggattggtgacgacagcc

SEQ ID NO: 66 - DNA, 20 bp, Artificial Sequence
  Synthetic oligonucleotide
attccttgggattggtgacg

SEQ ID NO: 67 - DNA, 20 bp, Artificial Sequence
  Synthetic oligonucleotide
cattccttgggattggtgac

SEQ ID NO: 68 - DNA, 20 bp, Artificial Sequence
  Synthetic oligonucleotide
tcattccttgggattggtga

SEQ ID NO: 69 - DNA, 20 bp, Artificial Sequence
  Synthetic oligonucleotide
agaagtccctcattccttgg

SEQ ID NO: 70 - DNA, 20 bp, Artificial Sequence
  Synthetic oligonucleotide
gtccactggaggagaagtcc

SEQ ID NO: 71 - DNA, 20 bp, Artificial Sequence
  Synthetic oligonucleotide
gtccttcaggtccactggag

SEQ ID NO: 72 - DNA, 20 bp, Artificial Sequence
  Synthetic oligonucleotide
catccctcgtccttcaggtc

SEQ ID NO: 73 - DNA, 20 bp, Artificial Sequence
  Synthetic oligonucleotide
tacatgaaatcccatccctc

SEQ ID NO: 74 - DNA, 20 bp, Artificial Sequence
  Synthetic oligonucleotide
cttggttacatgaaatccca

SEQ ID NO: 75 - DNA, 20 bp, Artificial Sequence
  Synthetic oligonucleotide
aatactcttggttacatgaa

SEQ ID NO: 76 - DNA, 20 bp, Artificial Sequence
  Synthetic oligonucleotide
ttagtaaaaatggaatactc

SEQ ID NO: 77 - DNA, 20 bp, Artificial Sequence
  Synthetic oligonucleotide
actgctttagtaaaaatgga

SEQ ID NO: 78 - DNA, 20 bp, Artificial Sequence
  Synthetic oligonucleotide
tgaaaacactgctttagtaa

SEQ ID NO: 79 - DNA, 20 bp, Artificial Sequence
  Synthetic oligonucleotide
tatgaggtgaaaacactgct

SEQ ID NO: 80 - DNA, 20 bp, Artificial Sequence
  Synthetic oligonucleotide
tagcatatgaggtgaaaaca

SEQ ID NO: 81 - DNA, 20 bp, Artificial Sequence
  Synthetic oligonucleotide
ttctaacatagcatatgagg

SEQ ID NO: 82 - DNA, 20 bp, Artificial Sequence
  Synthetic oligonucleotide
tggacttctaacatagcata

SEQ ID NO: 83 - DNA, 20 bp, Artificial Sequence
  Synthetic oligonucleotide
tctctgcctggacttctaac

SEQ ID NO: 84 - DNA, 20 bp, Artificial Sequence
  Synthetic oligonucleotide
ttattgtctctgcctggact

SEQ ID NO: 85 - DNA, 20 bp, Artificial Sequence
  Synthetic oligonucleotide
cctttcacaggaatgtttta

SEQ ID NO: 86 - DNA, 20 bp, Artificial Sequence
  Synthetic oligonucleotide
tgcctttcacaggaatgttt

SEQ ID NO: 87 - DNA, 20 bp, Artificial Sequence
  Synthetic oligonucleotide
gtgcctttcacaggaatgtt

SEQ ID NO: 88 - DNA, 20 bp, Artificial Sequence
  Synthetic oligonucleotide
aagtgcctttcacaggaatg

SEQ ID NO: 89 - DNA, 20 bp, Artificial Sequence
  Synthetic oligonucleotide
tgaaaagtgcctttcacagg

SEQ ID NO: 90 - DNA, 20 bp, Artificial Sequence
  Synthetic oligonucleotide
gcatgcagaggtgagtatac

SEQ ID NO: 91 - DNA, 20 bp, Artificial Sequence
  Synthetic oligonucleotide
agtccactcattcttggcag

SEQ ID NO: 92 - DNA, 20 bp, Artificial Sequence
  Synthetic oligonucleotide
tgctggactggtatttgtgt

SEQ ID NO: 93 - DNA, 20 bp, Artificial Sequence
  Synthetic oligonucleotide
ctacgggcaccggtgaatcc

SEQ ID NO: 94 - DNA, 20 bp, Artificial Sequence
  Synthetic oligonucleotide
ccggtgaatccaagtgtcct

SEQ ID NO: 95 - DNA, 20 bp, Artificial Sequence
  Synthetic oligonucleotide
gtcaaagttctagatgctgt

SEQ ID NO: 96 - DNA, 20 bp, Artificial Sequence
  Synthetic oligonucleotide
ttctagatgctgtccgaggc

SEQ ID NO: 97 - DNA, 20 bp, Artificial Sequence
  Synthetic oligonucleotide
ggccgtgcatgtgttcagaa

SEQ ID NO: 98 - DNA, 20 bp, Artificial Sequence
  Synthetic oligonucleotide
tgatgacacctgggagccat

SEQ ID NO: 99 - DNA, 20 bp, Artificial Sequence
  Synthetic oligonucleotide
ttgcctctgggaaaaccagt

SEQ ID NO: 100 - DNA, 20 bp, Artificial Sequence
  Synthetic oligonucleotide
ccagtgagtctggagagctg

SEQ ID NO: 101 - DNA, 20 bp, Artificial Sequence
  Synthetic oligonucleotide
tgcatgggctcacaactgag

SEQ ID NO: 102 - DNA, 20 bp, Artificial Sequence
  Synthetic oligonucleotide
actgaggaggaatttgtaga

SEQ ID NO: 103 - DNA, 20 bp, Artificial Sequence
  Synthetic oligonucleotide
tgtagaagggatatacaaag

SEQ ID NO: 104 - DNA, 20 bp, Artificial Sequence
  Synthetic oligonucleotide
gaaggcacttggcatctccc

SEQ ID NO: 105 - DNA, 20 bp, Artificial Sequence
  Synthetic oligonucleotide
ccattccatgagcatgcaga

SEQ ID NO: 106 - DNA, 20 bp, Artificial Sequence
  Synthetic oligonucleotide
ctactcctattccaccacgg

SEQ ID NO: 107 - DNA, 20 bp, Artificial Sequence
  Synthetic oligonucleotide
actcctattccaccacggct

SEQ ID NO: 108 - DNA, 20 bp, Artificial Sequence
  Synthetic oligonucleotide
ctattccaccacggctgtcg

SEQ ID NO: 109 - DNA, 20 bp, Artificial Sequence
  Synthetic oligonucleotide
accacggctgtcgtcaccaa

SEQ ID NO: 110 - DNA, 20 bp, Artificial Sequence
  Synthetic oligonucleotide
cacggctgtcgtcaccaatc

SEQ ID NO: 111 - DNA, 20 bp, Artificial Sequence
  Synthetic oligonucleotide
cggctgtcgtcaccaatccc

SEQ ID NO: 112 - DNA, 20 bp, Artificial Sequence
  Synthetic oligonucleotide
ggctgtcgtcaccaatccca

SEQ ID NO: 113 - DNA, 20 bp, Artificial Sequence
  Synthetic oligonucleotide
cgtcaccaatcccaaggaat

SEQ ID NO: 114 - DNA, 20 bp, Artificial Sequence
  Synthetic oligonucleotide
gtcaccaatcccaaggaatg

SEQ ID NO: 115 - DNA, 20 bp, Artificial Sequence
  Synthetic oligonucleotide
ccaaggaatgagggacttct

SEQ ID NO: 116 - DNA, 20 bp, Artificial Sequence
  Synthetic oligonucleotide
ggacttctcctccagtggac

SEQ ID NO: 117 - DNA, 20 bp, Artificial Sequence
  Synthetic oligonucleotide
ctccagtggacctgaaggac

SEQ ID NO: 118 - DNA, 20 bp, Artificial Sequence
  Synthetic oligonucleotide
gacctgaaggacgagggatg

SEQ ID NO: 119 - DNA, 20 bp, Artificial Sequence
  Synthetic oligonucleotide
gagggatgggatttcatgta

SEQ ID NO: 120 - DNA, 20 bp, Artificial Sequence
  Synthetic oligonucleotide
tgggatttcatgtaaccaag

SEQ ID NO: 121 - DNA, 20 bp, Artificial Sequence
  Synthetic oligonucleotide
ttcatgtaaccaagagtatt

SEQ ID NO: 122 - DNA, 20 bp, Artificial Sequence
  Synthetic oligonucleotide
tccatttttactaaagcagt

SEQ ID NO: 123 - DNA, 20 bp, Artificial Sequence
  Synthetic oligonucleotide
ttactaaagcagtgttttca

SEQ ID NO: 124 - DNA, 20 bp, Artificial Sequence
  Synthetic oligonucleotide
agcagtgttttcacctcata

SEQ ID NO: 125 - DNA, 20 bp, Artificial Sequence
  Synthetic oligonucleotide
tgttttcacctcatatgcta

SEQ ID NO: 126 - DNA, 20 bp, Artificial Sequence
  Synthetic oligonucleotide
cctcatatgctatgttagaa

SEQ ID NO: 127 - DNA, 20 bp, Artificial Sequence
  Synthetic oligonucleotide
tatgctatgttagaagtcca

SEQ ID NO: 128 - DNA, 20 bp, Artificial Sequence
  Synthetic oligonucleotide
gttagaagtccaggcagaga

SEQ ID NO: 129 - DNA, 20 bp, Artificial Sequence
  Synthetic oligonucleotide
agtccaggcagagacaataa

SEQ ID NO: 130 - DNA, 20 bp, Artificial Sequence
  Synthetic oligonucleotide
taaaacattcctgtgaaagg

SEQ ID NO: 131 - DNA, 20 bp, Artificial Sequence
  Synthetic oligonucleotide
aaacattcctgtgaaaggca

SEQ ID NO: 132 - DNA, 20 bp, Artificial Sequence
  Synthetic oligonucleotide
aacattcctgtgaaaggcac

SEQ ID NO: 133 - DNA, 20 bp, Artificial Sequence
  Synthetic oligonucleotide
cattcctgtgaaaggcactt

(19) United States
(12) Patent Application Publication
(10) Pub. No.: US 2011/0237646 A1
(43) Pub. Date: Sep. 29, 2011

(54) MODULATION OF TRANSTHYRETIN EXPRESSION FOR THE TREATMENT OF CNS RELATED DISORDERS

(75) Inventors: Richard Alan Smith, La Jolla, CA (US); Brett P. Monia, Encinitas, CA (US)

(73) Assignee: Isis Pharmaceuticals, Inc., Carlsbad, CA (US)

(21) Appl. No.: 13/056,295

(22) PCT Filed: Aug. 7, 2009
(86) PCT No.: PCT/US2009/053202

§ 371 Date: Jun. 15, 2011

Related U.S. Application Data
(60) Provisional application No. 61/087,146, filed on Aug. 7, 2008.
(60) Provisional application No. 61/092,698, filed on Aug. 28, 2008.

(51) Int. Cl. A61K 31/7088; A61K 31/7125; A61K 31/712; A61K 31/7115; A61P 25/00; A61P 25/28

(52) U.S. Cl. 514/44 A

(57) ABSTRACT

Compounds, compositions and methods are provided for modulating the expression of transthyretin in the brain, specifically the choroid plexus. The compositions comprise oligonucleotides, targeted to nucleic acid encoding transthyretin. Methods of using these compounds for modulation of transthyretin expression and for diagnosis and treatment of diseases and conditions associated with expression of transthyretin are provided.

### SEQUENCE LISTING

The present application is being filed along with a Sequence Listing in electronic format. The Sequence Listing is provided as a file entitled BIOL0104WOSEQ.txt, created on Aug. 7, 2009 which is 35 Kb in size. The information in the electronic format of the sequence listing is incorporated herein by reference in its entirety.

### FIELD OF THE INVENTION

The present invention provides compositions and methods for modulating the expression of transthyretin for the treatment of central nervous system related disorders. In particular, this invention relates to antisense compounds, particularly oligonucleotide compounds, which, in preferred embodiments, hybridize with nucleic acid molecules encoding transthyretin in the choroid plexus. Such compounds are shown herein to modulate the expression of transthyretin in the choroid plexus for the treatment of central nervous system related disorders. Further, such compounds are shown herein to modulate the expression of transthyretin in the choroid plexus for the treatment of central nervous system related disorders by cerebral intraventricular administration. Further, the invention provides methods of administering compounds by cerebral intraventricular administration for modulation in the choroid plexus.

### BACKGROUND OF THE INVENTION

Transthyretin (also known as TTR; prealbumin; prealbumin, thyroxine; PALB; TBPA; HST2651; amyloidosis 1, included; dysprealbuminemic euthyroidal hyperthyroxinemia, included; hyperthytoxinemia, dysprealbuminemic, included; hyperthytoxinemia, dystransthyretinemic, included; amyloid polyneuropathy, multiple forms, included; senile systemic amyloidosis, included) is a homotetrameric transport protein found in the extracellular fluids of vertebrates (Palha, Clin Chem Lab Med, 2002, 40, 1292-1300). Transthyretin was first identified as the major thyroid hormone carrier in the cerebrospinal fluid (CSF) and in the serum (Palha, Clin Chem Lab Med, 2002, 40, 1292-1300; Seibert, J. Biol. Chem., 1942, 143, 29-38). Transthyretin was cloned from adult human cDNA libraries and the gene was subsequently mapped to chromosome region 18q11.2-q12.1 (Mita et al., Biochem Biophys Res Commun, 1984, 124, 558-564; Sparkes et al., Hum Genet, 1987, 75, 151-154; Whitehead et al., Mol Biol Med, 1984, 2, 411-423).

The liver and the choroid plexus are the primary sites of transthyretin synthesis in humans (Palha, Clin Chem Lab Med, 2002, 40, 1292-1300). Transthyretin that is synthesized in the liver is secreted into the blood, whereas transthyretin originating in the choroid plexus is destined for the CSF. In the choroid plexus, transthyretin synthesis represents about 20% of total local protein synthesis and as much as 25% of the total CSF protein (Dickson et al., J Biol Chem, 1986, 261, 3475-3478).

Transthyretin is associated with both local and systemic amyloidosis, a disorder characterized by extracellular systemic deposition of mutated or wild-type transthyretin as amyloid fibrils (Cornwell et al., Biochem Biophys Res Commun, 1988, 154, 648-653; Saraiva et al., J Clin Invest, 1984, 74, 104-119; Yazaki et al., Muscle Nerve, 2003, 28, 438-442), leading to organ dysfunction and death. Senile systemic amyloidosis (SSA) is a sporadic disorder resulting from the extracellular deposition of wild-type transthyretin fibrils in cardiac and other tissues. Inherited mutations in transthyretin are causative defects for both familial amyloid polyneuropathy (FAP) and familial amyloid cardiomyopathy (FAC). Disease results from neurodegeneration and/or organ dysfunction associated with transthyretin amyloid fibril deposits in a variety of tissues, particularly the peripheral and central nervous system and heart. Over 80 mutations in transthyretin are associated with familial amyloidotic polyneuropathy and cardiomyopathy. In most of these cases, inheritance is autosomal dominant (Reixach et al., Proc Natl Acad Sci USA, 2004, 101, 2817-2822). Jiang et al (Jiang et al., Proc Natl Acad Sci USA, 2001, 98, 14943-14948) demonstrated that the variant with a valine to isoleucine mutation at amino acid 122 (Val122Ile), which is among the most common amyloidogenic mutations worldwide, increases the velocity of rate-limiting tetramer dissociation, thereby resulting in accelerated amyloidogenesis. This finding suggests the possibility that treatments for transthyretin-related amyloidoses may include small molecules that stabilize the tetrameric form (Adamski-Werner et al., J Med Chem, 2004, 47, 355-374; Altland and Winter, Neurogenetics, 1999, 2, 183-188). Small molecule stabilizers were also shown to be of use in preventing the formation of amyloid fibrils of the wildtype transthyretin (Reixach et al., Proc Natl Acad Sci USA, 2004, 101, 2817-2822). Other common transthyretin mutations associated with amyloidosis include Val30Met and Glu61Lys. In vitro studies have shown success using ribozymes to specifically target and inhibit the expression of the Glu61Lys and Val30Met variants (Propsting et al., Biochem Biophys Res Commun, 1999, 260, 313-317; Tanaka et al., J Neurol Sci, 2001, 183, 79-84). Single-stranded oligonucleotides have been used both in vitro and in vivo to correct single-base mutation (Val30Met) in transthyretin to the wild-type form through targeted recombination (Nakamura et al., Gene Ther, 2004). The success of this therapy was limited, however, with gene conversion rates of 11% in vitro and 9% in vivo. These levels are not sufficient for suppression of the variant transthyretin in clinical terms (Nakamura et al., Gene Ther, 2004). Other treatment options for transthyretin-associated familial amyloidosis include surgical removal of fibril deposits and in some cases liver transplant. The latter is a gene therapy approach introducing a wild-type gene into the patient. The effectiveness of transplantation in treating familial amyloid disease is limited by continued production of mutant transthyretin by the choroid plexus. Transplant options are non-viable for SSA patients, since wild-type transthyretin fibrils are deposited.

Consequently, there remains an unmet need for agents capable of effectively modulating transthyretin expression (Nakamura et al., Gene Ther, 2004; Tanaka et al., J Neurol Sci, 2001, 183, 79-84) particularly for the treatment of amyloidosis and central nervous system related diseases and disorders.

Antisense technology is an effective means of reducing the expression of specific gene products and therefore is uniquely useful in a number of therapeutic, diagnostic, and research applications for the modulation of transthyretin expression. The present invention provides compositions and methods for modulating transthyretin expression for the treatment of central nervous system related disorders.

### SUMMARY OF THE INVENTION

The present invention is directed to antisense compounds, especially nucleic acid and nucleic acid-like oligomers, which are targeted to a nucleic acid encoding transthyretin, and which modulate the expression of transthyretin in the central nervous system. Pharmaceutical and other compositions comprising the compounds of the invention are also provided. Further provided, are methods of screening for modulators of transthyretin and methods of modulating the expression of transthyretin in cells, tissues or animals comprising contacting said cells, tissues or animals with one or more of the compounds or compositions of the invention. Methods of treating an animal, particularly a human, suspected of having or being prone to diseases or conditions associated with expression of transthyretin are also set forth herein. Such methods comprise administering a therapeutically or prophylactically effective amount of one or more of the compounds or compositions of the invention to the person in need of treatment.

### BRIEF DESCRIPTION OF THE DRAWINGS

FIG. 1: Subcutaneous administration of transthyretin antisense oligonucleotides lowers liver mRNA in transgenic mice. Transthyretin liver mRNA levels for animals treated with subcutaneous antisense oligonucleotides ISIS 304309 at 25 μg/kg were 14±3 percent of controls (P<0.05).

FIG. 2: Cerebral intraventricular administration of transthyretin antisense oligonucleotides lowers human-transthyretin mRNA in transgenic mice. transthyretin choroid mRNA levels for animals treated with antisense oligonucleotides ISIS 304309 50 μg/day were 61±5 percent of controls and 49±5 percent of control for animals treated with antisense oligonucleotides 75 μg/day (P<0.05).

FIG. 3: Representative section of choroid plexus from saline treated animal stained with anti-human transthyretin shows marked staining in cytoplasm of epithelial cells. TTR is mainly localized in the cytoplasm apical to the nuclei of epithelial cells.

FIG. 4: Representative section of choroid plexus from animal treated with intrathecal antisense oligonucleotides ISIS 304309 50 μg/day stained with anti-human transthyretin showing little staining of epithelial cells.

FIG. 5: Represents twelve transthyretin mutations that have been reported to be associated with clinically significant amyloid deposits in leptomeninges and casuclar structures of the brain.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### DEFINITIONS

Unless specific definitions are provided, the nomenclature utilized in connection with, and the procedures and techniques of analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to the 5′ position. A 5-methylcytosine is a modified nucleobase.

“Active pharmaceutical ingredient” means the substance or substances in a pharmaceutical composition that provides a desired effect.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration.

“Administering” means providing a pharmaceutical agent to an individual, and includes, but is not limited to administering by a medical professional and self-administering.

“Amelioration” refers to a lessening of at least one indicator, sign, or symptom of an associated condition or disease. The severity of indicators may be determined by subjective or objective measures, which are known to those skilled in the art.

“Amyloidosis” is a disorder resulting from abnormal protein (amyloid or amyloid fibril) deposits in body tissues.

“Animal” refers to a human or non-human animal, including, but not limited to, mice, rats, rabbits, dogs, cats, pigs, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense compound” means an oligomeric compound that is capable of undergoing hybridization to a target nucleic acid through hydrogen bonding.

“Antisense inhibition” means reduction of a target nucleic acid levels in the presence of an antisense compound complementary to a target nucleic acid compared to target nucleic acid levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furosyl ring modified by the bridging of two non-geminal ring atoms. A bicyclic sugar is a modified sugar.

“Cap structure” or “terminal cap moiety” means chemical modifications, which have been incorporated at either terminus of an antisense compound.

“Central nervous system (CNS)” refers to the vertebrate nervous system which is enclosed in meninges. It contains the majority of the nervous system, and consists of the brain (in vertebrates which have brains), and the spinal cord. The CNS is contained within the dorsal cavity, with the brain within the cranial cavity, and the spinal cord in the spinal cavity. The brain is also protected by the skull, and the spinal cord is, in vertebrates, also protected by the vertebrae.

“Central nervous system related disorders” refers to all disorders related to conditions of the central nervous system that cause disease or disorder. For example, a central nervous system related disorder includes, but is not limited to, a transthyretin amyloid disease such as leptomeningeal amyloidosis or familial amyloid polyneuropathy (FAP).

“Chimeric antisense compound” means an antisense compound that has at least 2 chemically distinct regions, each position having a plurality of subunits.

“Choroid plexus” is the area on the ventricles of the brain where cerebrospinal fluid (CSF) is produced.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses administration in parallel or sequentially.

“Complementarity” means the capacity for pairing between nucleobases of a first nucleic acid and a second nucleic acid.

“Contiguous nucleobases” means nucleobases immediately adjacent to each other.

“Diluent” means an ingredient in a composition that lacks pharmacological activity, but is pharmaceutically necessary or desirable. For example, in agents that are injected the diluent may be a liquid, e.g. saline solution.

“Dose” means a specified quantity of a pharmaceutical agent provided in a single administration, or

in a specified time period. In certain embodiments, a dose may be administered in one, two, or more boluses, tablets, or injections. For example, in certain embodiments, where parenteral administration is desired, the desired dose requires a volume not easily accommodated by a single injection. In certain embodiments, two or more injections may be used to achieve the desired dose. In certain embodiments, a dose may be administered in one, two, or more injections to minimize injection site reaction in an individual. In certain embodiments, the pharmaceutical agent is administered by infusion over an extended period of time or continuously. Doses may be stated as the amount of pharmaceutical agent per hour, day, week or month.

“Effective amount” in the context of modulating an activity or of treating or preventing a condition means the administration of that amount of active ingredient to a subject in need of such modulation, treatment or prophylaxis, either in a single dose or as part of a series, that is effective for modulation of that effect, or for treatment or prophylaxis or improvement of that condition. The effective amount will vary depending upon the health and physical condition of the subject to be treated, the taxonomic group of subjects to be treated, the formulation of the composition, the assessment of the medical situation, and other relevant factors.

“Familial amyloidosis” or “hereditary amyloidosis” is a form of inherited amyloidosis.

“Familial amyloid polyneuropathy” or “FAP” is a neurodegenerative genetically transmitted disorder, characterized by systemic depositions of amyloid variants of tranthyretin proteins, causing progressive sensory and motorial polyneuropathy.

“Fully complementary” means each nucleobase of a first nucleic acid has a complementary nucleobase in a second nucleic acid. In certain embodiments, a first nucleic acid is an antisense compound and a target nucleic acid is a second nucleic acid. In certain embodiments, an antisense oligonucleotide is a first nucleic acid and a target nucleic acid is a second nucleic acid.

“Gapmer” means an antisense compound in which an internal position having a plurality of nucleotides that supports RNaseH cleavage is positioned between external regions having one or more nucleotides that are chemically distinct from the nucleosides of the internal region. A “gap segment” means the plurality of nucleotides that make up the internal region of a gapmer. A “wing segment” means the external region of a gapmer.

“Gap-widened” means an antisense compound has a gap segment of 12 or more contiguous 2′-deoxyribonucleotides positioned between and immediately adjacent to 5′ and 3′ wing segments having from one to six nucleotides having modified sugar moieties.

“Hereditary Transthyretin (TTR) amyloidosis” is a systemic disease caused by mutations in transthyretin, a plasma transport protein for thyroxine and vitamin A. It is most frequently associated with peripheral neuropathy and restrictive cardiomyopathy, but amyloid deposits in blood vessel walls and connective tissue structures throughout the body often cause dysfunction of other organ systems. Gastrointestinal motility abnormalities are common in this disease with constipation, diarrhea and early satiety from delayed gastric-emptying. Connective tissue deposits of amyloid in the wrist may cause carpal tunnel syndrome. Amyloid deposits in spinal blood vessels and surrounding structures cause spinal stenosis with symptoms of claudication.

“Hybridization” means the annealing of complementary nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

“Immediately adjacent” means there are no intervening nucleotides between the immediately adjacent elements.

“Individual” means a human or non-human animal selected for treatment or therapy.

“Internucleoside linkage” refers to the chemical bond between nucleosides.

“Intracerebroventricular administration” or “cerebral intraventricular administration” or “cerebral ventricular administration” means administration through injection or infusion into the ventricular system of the brain.

“Intraperitoneal administration” means administration to the peritoneal cavity.

“Intrathecal administration” means administration through injection or infusion into the cerebrospinal fluid bathing the spinal cord and brain.

“Intravenous administration” means administration into a vein.

“Intraventricular administration” means administration into the ventricles of either the brain or heart.

“Leptomeningeal” means having to do with the leptomeninges, the two innermost layers of tissues that cover the brain abd spinal cord. “Leptomeningeal amyloidosis” refers to amyloidosis of the leptomeninges resulting from transthyretin amyloid deposition within the leptomeninges.

“Linked nucleosides” means adjacent nucleosides which are bonded together.

“Mismatch” or “non-complementary nucleobase” means a nucleobase of first nucleic acid that is not capable of pairing with the corresponding nucleobase of a second or target nucleic acid.

“Modified internucleoside linkage” refers to a substitution and/or any change from a naturally occurring internucleoside bond (i.e. a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Modified nucleotide” means a nucleotide having, independently, a modified sugar moiety, modified internucleoside linkage, or modified nucleobase. A “modified nucleoside” means a nucleotide having, independently, a modified sugar moiety or modified nucleobase.

“Modified oligonucleotide” means an oligonucleotide comprising a modified internucleoside linkage, a modified sugar, and/or a modified nucleobase.

“Modified sugar” refers to a substitution and/or any change from a natural sugar.

“Motif” means the pattern of unmodified and modified nucleosides in an antisense compound.

“Naturally occurring internucleoside linkage” means a 3′ to 5′ phosphodiester linkage.

“Natural sugar” means a sugar found in DNA (2′-H) or RNA (2′-OH).

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase sequence” means the order of contiguous nucleobases independent of any sugar, linkage, and/or nucleobase modification.

“Nucleoside” means a nucleobase linked to a sugar.

“Nucleotide” means a nucleoside having a phosphate group covalently linked to the sugar portion of the nucleoside.

“Oligomeric compound” means a polymer of linked monomeric subunits which is capable of hybridizing to at least a region of a nucleic acid molecule.

“Oligonucleoside” means an oligonucleotide in which the internucleoside linkages do not contain a phosphorus atom.

“Oligonucleotide” means a polymer of linked nucleosides each of which can be modified or unmodified, independent one from another.

“Parenteral administration,” means administration through injection or infusion. Parenteral administration includes but is not limited to, intravenous, intraarterial, subcutaneous, intraperitoneal, intramuscular injection or infusion, or intracranial, e.g., intracerebral administration, intrathecal administration, intraventricular administration, ventricular administration, intracerebroventricular administration, cerebral intraventricular administration or cerebral ventricular administration. “Peptide” means a molecule formed by linking at least two amino acids by amide bonds. Without limitation, as used herein, “peptide” refers to polypeptides and proteins.

“Pharmaceutical agent” means a substance that provides a therapeutic benefit when administered to an individual. For example, in certain embodiments, an antisense oligonucleotide targeted to transthyretin is pharmaceutical agent.

“Pharmaceutical composition” means a mixture of substances suitable for administering to an individual. For example, a pharmaceutical composition may comprise one or more antisense oligonucleotides and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate internucleoside linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prodrug” means a therapeutic agent that is prepared in an inactive form that is converted to an active form (i.e., drug) within the body or cells thereof by the action of endogenous enzymes or other chemicals and/or conditions.

“Side effects” means physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand.

“Subcutaneous administration” means administration just below the skin.

“Targeted” or “targeted to” means having a nucleobase sequence that will allow hybridization of an antisense compound to a target molecule to induce a desired effect. In certain embodiments, a desired effect is reduction of a target nucleic acid. In certain embodiments, a desired effect is reduction of transthyretin mRNA or protein expression.

“Targeting” means the process of design and selection of an antisense compound that will specifically hybridize to a target nucleic acid and induce a desired effect.

“Target nucleic acid,” “target RNA,” “target RNA transcript” and “nucleic acid target” all mean a nucleic acid capable of being targeted by antisense compounds.

“Target region” means a portion of a target nucleic acid to which one or more antisense compounds is targeted.

“Target segment” refers to a smaller portion or sub-portion of a region within a target nucleic acid. A target segment can be the sequence of nucleotides of a target nucleic acid to which an antisense compound is targeted.

“Therapeutically effective amount” means an amount of a pharmaceutical agent that provides a therapeutic benefit to an individual.

“Transthyretin-specific inhibitor” or “Transthyretin inhibitor” means any compound capable of decreasing Transthyretin mRNA or protein expression. Examples of such compounds include a nucleic acid, a peptide, an antibody, or a histone deacetylase inhibitor.

“Transthyretin specific modulator” or “transthyretin modulator” means any compound capable of increasing or decreasing transthyretin mRNA or protein expression.

“Transthyretin amyloid disease”, as used herein, is any pathology or disease associated with dysfunction or dysregulation of transthyretin that result in formation of transthyretin-containing amyloid fibrils. Transthyretin amyloid disease includes, but is not limited to, leptomeningeal amyloidosis or familial amyloid polyneuropathy (FAP).

“Unmodified nucleotide” means a nucleotide composed of naturally occurring nucleobases, sugar moieties and internucleoside linkages. In certain embodiments, an unmodified nucleotide is an RNA nucleotide (i.e., β-D-ribonucleosides) or a DNA nucleotide (i.e., β-D-deoxyribonucleoside).

### Certain Embodiments

Transthyretin amyloidosis or transthyretin amyloid disease is an autosomal dominant Mendellian disease, and most affected individuals are heterozygous for one of approximately 100 disease associated transthyretin mutations. Plasma transthyretin is mainly synthesized by the liver. However, transthyretin is also synthesized by the choroid plexus of the brain and the retinal pigment epithelium of the eye. Approximately 25 percent of amyloid transthyretin mutations are associated with deposits in the vitreous of the eye and this has been hypothesized to be the result of local synthesis of transthyretin by the retinal pigment epithelium. Twelve transthyretin mutations have been reported to be associated with clinically significant amyloid deposits and/or fibril formation in leptomeninges and vascular structures of the brain and may cause subarachnoid or intracerebral hemorrhage, seizures, hydrocephalus or dementia (FIG. 5). Leptomeningeal amyloid deposits and fibril formation have been shown to be derived from transthyretin synthesized by the choroid plexus. The present invention provides for a method of reducing TTR expression in the choroid plexus, by administrating TTR ASOs to brain. The present invention shows that the reduction of TTR in the choid plexus could result in the reduction of amyloid deposits and fibril formation.

Antisense oligonucleotides (ASO) when administered to animals by subcutaneous, intravenous or intraperitoneal injection distribute effectively to a number of organs including liver, kidney, bone marrow and spleen, with virtually no antisense oligonucleotide accumulating in brain. However, little antisense oligonucleotide is detectable in regions within or surrounding the choroid plexus following systemic administration. It has been previously shown that transthyretin specific antisense oligonucleotides, when administered to can significantly suppress hepatic synthesis transthyretin. While transthyretin plasma levels and hepatic transthyretin mRNA levels could be suppressed as much as 80 percent of baseline values, immunohistochemical analysis of CNS tissue indicated no effect on choroid plexus transthyretin. However, as provided herein, local administration of transthyretin antisense oligonucleotide to brain via ventricular injection results in a dose-dependent reduction in transthyretin levels in brain. The present invention also provides transthyretin inhibitors as described herein for the use in treating or preventing central nervous system related disorders by cerebral intraventricular administration.

The present invention provides a transthyretin inhibitor as described herein for use in treating or preventing a central nervous system related disorder as described herein. For example, the invention provides a transthyretin inhibitor as described herein for use in treating or preventing transthyretin amyloid disease. For example, the invention provides a transthyretin inhibitor as described herein for use in treating or preventing transthyretin amyloidosis. For example, the invention also provides a transthyretin inhibitor as described herein for use in treating or preventing leptomeningeal amyloidosis.

It has been previously shown that subcutaneous administration of antisense oligonucleotides specific for human transthyretin can significantly suppress the synthesis of transthyretin in the liver in mice transgenic for a human amyloid associated transthyretin mutation (Ile84Ser). This previous approach to treatment was based on the premise that restricting the availability of an amyloid precursor protein would inhibit fibril formation. However, amyloid despoits and fibril formation in the brain were still present in this treatment. The present invention shows that the reduction of TTR in the choid plexus could result in the reduction of amyloid deposits and fibril formation. Previous clinical experience for treatment of patients with immunoglobulin light chain (AL) amyloidosis has shown that chemotherapy can help stop the production of monoclonal Ig protein. As well as in patients with reactive (AA) amyloidosis, chemotherapy seems to reduce systemic inflammation and, therefore, plasma serum amyloid A (SAA) levels. However, it is unlikely that chemotherapy will be proven to significantly inhibit the progression of leptomeningeal deposition of transthyretin amyloid. The present invention shows that the reduction of TTR in the choid plexus could result in the reduction of amyloid deposits and fibril formation. Furthermore, due to the reduction in TTR expression in the choid plexus, the present invention could result in the reduction progression of leptomeningeal deposition of transthyretin amyloids.

Another treatment that has been previously shown is orthotopic liver transplantation (OLT) for patients with amyloidogenic transthyretin mutations essentially eliminates mutant transthyretin from the plasma and has been shown to be an effective treatment for many patients with transthyretin amyloidosis. However, recently it has now been shown that in a significant number of patients who received OLT, the disease can progress with continued amyloid fibril formation from normal transthyretin. In addition, vitreous opacities may occur after OLT and presumably are the result of mutant transthyretin synthesized by retinal pigment epithelium. Similarly, the choroid plexus epithelium may continue to synthesize mutant transthyretin after OLT. While one report of Tyr114Cys patients suggests a beneficial effect from OLT, it is unlikely that OLT will be proven to significantly inhibit the progression of leptomeningeal deposition of transthyretin amyloid.

The present invention herein provides for a treatment for transthyretin amyloidosis, such as leptomeningeal amyloidosis. The present invention also provides for methods of inhibiting TTR expression in the choroid plexus by administering TTR ASOs. While human plasma TTR levels and hepatic TTR mRNA levels could be suppressed with subcutaneous administration of TTR ASOs, immunohistochemical analysis of CNS tissue indicates no effect on choroid plexus TTR. The present invention provides for a method of local administration of TTR ASOs to brain via intracerebral ventricular injection that results in a dose dependent reduction of TTR expression by the choroid plexus.

The present invention provides, as shown herein, subcutaneous administration of human transthyretin specific antisense oligonucleotides significantly suppressed hepatic transthyretin synthesis but gave no significant suppression of human transthyretin expression by the choroid plexus epithelium. However, administration of transthyretin antisense oligonucleotides via the cerebral ventricular system did significantly suppress choroid expression of transthyretin as measured by transthyretin mRNA levels. Immunohistochemical staining of choroid plexus with anti-human transthyretin was also consistent with suppression of transthyretin synthesis following cerebral intraventricular administration of antisense oligonucleotides, but considerable variability was noted amongst treated animals, perhaps a result of tissue sampling. In addition, immunohistochemistry may not resolve differences in the magnitude of protein expression that in this instance, are likely to be 25-40 percent of normal. As expected, cerebral intraventricular administration of antisense oligonucleotides had no effect on hepatic human transthyretin mRNA levels.

The present invention provides a transthyretin inhibitor as described herein for use in treating, ameliorating, and/or preventing a central nervous system related disorder, or CNS disease, as described herein. For example, the invention provides a transthyretin inhibitor as described herein for use in treating, ameliorating, and/or preventing central nervous system related disease or disorders; for example, transthyretin amyloid disease.

The present invention also provides a transthyretin inhibitor as described herein for use in treating, ameliorating, and/or preventing transthyretin amyloid disease, such as, but not limited to, leptomeningeal amyloidosis.

In any of the methods described herein, a human subject (e.g., a human patient) can have, or be at risk of developing (e.g., have a genetic predisposition to developing), a transthyretin amyloid disease. Transthyretin amyloid disease includes, but is not limited to, leptomeningeal amyloidosis or familial amyloid polyneuropathy (FAP).

The present invention also provides the use of a transthyretin inhibitor as described herein in the manufacture of a medicament for treating, ameliorating, and/or preventing a central nervous system related disorder as described herein. For example, the invention provides the use of a transthyretin inhibitor as described herein in the manufacture of a medicament for treating, ameliorating, and/or preventing a transthyretin amyloid disease. The transthyretin amyloid disease includes, but is not limited to, leptomeningeal amyloidosis or familial amyloid polyneuropathy (FAP).

The present invention also provides a transthyretin inhibitor as described herein for use in treating, ameliorating, and/or preventing transthyretin amyloid disease, such as, but not limited to, leptomeningeal amyloidosis.

In any of the methods described herein, a human subject (e.g., a human patient) can have, or be at risk of developing (e.g., have a genetic predisposition to developing), a transthyretin amyloid disease. Transthyretin amyloid disease includes, but is not limited to, leptomeningeal amyloidosis or familial amyloid polyneuropathy (FAP).

Leptomeningeal transthyretin amyloidosis is a prominent feature of several of the transthyretin amyloidogenic mutations. It may be the principal clinical feature, as in patients with the Tyr114Cys, Val30Gly, and Glu18Gly mutations where systemic amyloid deposition is sometimes mild, or less life threatening in patients with other mutations where cardiomyopathy or nephropathy dictate survival (e.g. Val30Met). Dementia is a prominent feature of the Glu18Gly, Tyr114Cys and Val30Gly mutations, and cerebral hemorrhage is often the cause of death.

The present invention provides methods of treatment with transthyretin specific antisense oligonucleotidess by subcutaneous injection proves to be effective for systemic transthyretin amyloidoses, however it is not likely to alter the course of disease in patients who have leptomeningeal amyloidosis as their life threatening manifestation of the disease. Administration of specific transthyretin antisense oligonucleotides directly into the cerebral ventricular system, however, significantly suppresses transthyretin expression by the choroid plexus epithelium and may offer an effective treatment for this devastating disease.

The present invention also provides the use of a transthyretin inhibitor as described herein in the manufacture of a medicament for treating or preventing a central nervous system related disease or disorder as described herein. For example, the invention provides the use of a transthyretin inhibitor as described herein in the manufacture of a medicament for treating or preventing amyloidosis. For example, the invention provides the use of a transthyretin inhibitor as described herein in the manufacture of a medicament for treating or preventing leptomeningeal amyloidosis.

The invention also provides a transthyretin inhibitor as described herein for reducing transthyretin mRNA levels, e.g. for reducing transthyretin mRNA levels in a subject having elevated transthyretin mRNA levels. The present invention also provides the use of a transthyretin inhibitor as described herein in the manufacture of a medicament for reducing transthyretin mRNA levels, e.g. for reducing transthyretin mRNA levels in a subject having elevated transthyretin mRNA levels.

The invention also provides a transthyretin inhibitor as described herein for reducing transthyretin mRNA levels in the choroid plexus, e.g. for reducing transthyretin mRNA levels in the choroid plexus in a subject having elevated transthyretin mRNA levels in the choroid plexus. The present invention also provides the use of a transthyretin inhibitor as described herein in the manufacture of a medicament for reducing transthyretin mRNA levels in the choroid plexus, e.g. for reducing transthyretin mRNA levels in the choroid plexus in a subject having elevated transthyretin mRNA levels in the choroid plexus.

The invention also provides a transthyretin inhibitor as described herein for reducing transthyretin mRNA levels in the choroid plexus by cerebral intraventricular administration, e.g. for reducing transthyretin mRNA levels in the choroid plexus in a subject having elevated transthyretin mRNA levels in the choroid plexus by cerebral intraventricular administration. The present invention also provides the use of a transthyretin inhibitor as described herein in the manufacture of a medicament for reducing transthyretin mRNA levels in the choroid plexus by cerebral intraventricular administration, e.g. for reducing transthyretin mRNA levels in the choroid plexus in a subject having elevated transthyretin mRNA levels in the choroid plexus by cerebral intraventricular administration.

The invention also provides a transthyretin inhibitor as described herein for use in treating or preventing a central nervous system related disease or disorder as described herein by combination therapy with an additional therapy as described herein.

The invention also provides a pharmaceutical composition comprising a transthyretin inhibitor as described herein in combination with an additional therapy as described herein.

The invention also provides the use of a transthyretin inhibitor as described herein in the manufacture of a medicament for treating or preventing a central nervous system related disease or disorder as described herein by combination therapy with an additional therapy as described herein.

The invention also provides the use of a transthyretin inhibitor as described herein in the manufacture of a medicament for treating or preventing a central nervous system related disease or disorder as described herein in a patient who has previously been administered an additional therapy as described herein.

The invention also provides the use of a transthyretin inhibitor as described herein in the manufacture of a medicament for treating or preventing a central nervous system related disease or disorder as described herein in a patient who is subsequently to be administered an additional therapy as described herein.

The invention also provides a kit for treating or preventing a central nervous system related disease or disorder as described herein, said kit comprising:

(i) a transthyretin inhibitor as described herein; and
(ii) administered intraventricularly.

The invention also provides a kit for treating or preventing a central nervous system related disease or disorder as described herein, said kit comprising:
(i) a transthyretin inhibitor as described herein
(ii) administered intraventricularlry; and
(iii) an additional therapy as described herein

A kit of the invention may further include instructions for using the kit to treat or prevent a central nervous system related disease or disorder as described herein by combination therapy as described herein.

A kit of the invention may further include instructions for using the kit to treat, ameliorate, and/or prevent a central nervous system related disease or disorder and/or transthyretin amyloid diesease, as described herein by combination therapy as described herein.

Antisense compounds described herein may comprise an oligonucleotide consisting of 12 to 30 linked nucleosides targeted to a transthyretin nucleic acid.

Also described herein are methods for treating, ameliorating, and/or preventing an animal having central nervous system related disease or disorder and/or transthyretin amyloid diesease.

In certain embodiments, the method comprises inhibiting transthyretin in the brain of an animal by administering a modified oligonucleotide consisting of 12 to 30 linked nucleosides, wherein the modified oligonucleotide is complementary to human transthyretin; and thereby inhibiting transthyretin in the brain of an animal.

In certain embodiments, the transthyretin inhibitor is a nucleic acid.

In certain embodiments the nucleic acid is a modified oligonucleotide.

In certain embodiments the modified oligonucleotide is a single-stranded oligonucleotide.

In certain embodiments the nucleobase sequence of the modified oligonucleotide is 100% complementary to human transthyretin.

In certain embodiments at least one internucleoside linkage is a modified internucleoside linkage.

In certain embodiments each internucleoside linkage is a phosphorothioate internucleoside linkage.

In certain embodiments at least one nucleoside comprises a modified sugar.

In certain embodiments at least one modified sugar is a bicyclic sugar.

In certain embodiments at least one modified sugar comprises a 2′-O-methoxyethyl.

In certain embodiments at least one nucleoside comprises a modified nucleobase.

In certain embodiments, the method comprises identifying an animal having a central nervous system disorder by administering to the brain of the animal having a central nervous system disorder a therapeutically effective amount of a transthyretin inhibitor.

In certain embodiments the central nervous system disorder is a transthyretin amyloid disease.

In certain embodiments the transthyretin amyloid disease consists of leptomeningeal amyloidosis or familial amyloid polyneuropathy.

In certain embodiments, the method comprises reducing amyloid fibril formation in an animal comprising by administering to the brain of the animal a therapeutically effective amount of a compound comprising a modified oligonucleotide consisting of 12 to 30 linked nucleosides, wherein the modified oligonucleotide is complementary to human transthyretin; and thereby reducing amyloid fibril formation in an animal.

In certain embodiments, the animal has a central nervous system related disorder.

In certain embodiments, the central nervous system related disorder is transthyretin amyloid disease.

In certain embodiments, the transthyretin amyloid disease consists of leptomeningeal amyloidosis or familial amyloid polyneuropathy.

In certain embodiments the administering results in a 20% reduction of amyloid fibril formation.

In certain embodiments, the administering results in a 30% reduction of amyloid fibril formation.

In certain embodiments the administering results in a 40% reduction of amyloid fibril formation.

In certain embodiments the administering results in a 50% reduction of amyloid fibril formation.

In certain embodiments the administering results in a 60% reduction of amyloid fibril formation.

In certain embodiments the administering results in a 70% reduction of amyloid fibril formation.

In certain embodiments the administering results in an 80% reduction of amyloid fibril formation.

In certain embodiments the administering results in a 90% reduction of amyloid fibril formation.

In certain embodiments the administering results in a 100% reduction of amyloid fibril formation.

In certain embodiments the administering results in a 20% reduction of amyloid deposits.

In certain embodiments, the administering results in a 30% reduction of amyloid deposits.

In certain embodiments the administering results in a 40% reduction of amyloid deposits.

In certain embodiments the administering results in a 50% reduction of amyloid deposits.

In certain embodiments the administering results in a 60% reduction of amyloid deposits.

In certain embodiments the administering results in a 70% reduction of amyloid deposits.

In certain embodiments the administering results in an 80% reduction of amyloid deposits.

In certain embodiments the administering results in a 90% reduction of amyloid deposits.

In certain embodiments the administering results in a 100% reduction of amyloid deposits.

In certain embodiments, the method comprises preventing, ameliorating, or treating of a central nervous system disorder in an animal comprising by identifying an animal having a central nervous system disorder and administering to the brain of the animal a therapeutically effective amount of a compound comprising a modified oligonucleotide consisting of 12 to 30 linked nucleosides, wherein the modified oligonucleotide is complementary to human transthyretin; and thereby preventing, ameliorating, or treating of a central nervous system disorder in an animal.

In certain embodiments, the central nervous system disorder is a transthyretin amyloid disease.

In certain embodiments, the transthyretin amyloid disease consists of leptomeningeal amyloidosis or familial amyloid polyneuropathy.

In certain embodiments, the method comprises inhibiting transthyretin in the choroid plexus of an animal comprising by administering a modified oligonucleotide consisting of 12 to 30 linked nucleosides, wherein the modified oligonucleotide is complementary to human transthyretin; and thereby inhibiting transthyretin in the brain of an animal.

In certain embodiments the administering comprises intracranial administration.

In certain embodiments the intracranial administration can be intracerebral administration, intrathecal administration, intraventricular administration, ventricular administration, intracerebroventricular administration, cerebral intraventricular administration or cerebral ventricular administration.

In certain embodiments, the method comprises identifying an animal having a central nervous system related disease or disorder and/or transthyretin amyloid diesease, and administering to the animal having central nervous system related disease or disorder and/or transthyretin amyloid diesease, a therapeutically effective amount of a transthretin inhibitor.

In certain embodiments, the method comprises identifying an animal having leptomeningeal amyloidosis and administering to the animal having a leptomeningeal amyloidosis a therapeutically effective amount of a tranthyretin inhibitor.

In certain embodiments, the transthyretin inhibitor is a nucleic acid

In certain embodiments, the nucleic acid is a modified oligonucleotide.

In certain embodiments, the modified oligonucleotide may be a single-stranded or double-stranded oligonucleotide. The modified oligonucleotide may be 70, 75, 80, 85, 90, 95, or 100% complementary to a human tranthyretin nucleic acid.

The modified oligonucleotide may have at least one modified internucleoside linkage. The internucleoside linkage may be a phosphorothioate internucleoside linkage.

The modified oligonucleotide may have at least one modified sugar. The modified sugar may be a bicyclic sugar. The modified sugar may comprise a 2′-O-methoxyethyl.

The modified oligonucleotide may comprise at least one nucleoside having a modified nucleobase.

The modified oligonucleotide may have the nucleobase sequence of any of SEQ ID NOs: 12-133.

In certain embodiments, the method comprises identifying an animal having a central nervous system related disease or disorder and administering to the animal having central nervous system related disease or disorder a therapeutically effective amount of a compound comprising a modified oligonucleotide consisting of 12 to 30 linked nucleosides, wherein the modified oligonucleotide is complementary to human tranthyretin.

In certain embodiments, the method comprises identifying an animal having a transthyretin amyloid diesease and administering to the animal having transthyretin amyloid diesease a therapeutically effective amount of a compound comprising a modified oligonucleotide consisting of 12 to 30 linked nucleosides, wherein the modified oligonucleotide is complementary to human tranthyretin.

In certain embodiments, transthyretin amyloid diesease, includes but is not limited to, familial amyloid polyneuropathy, senile systemic amyloidosis, or leptomeningeal amyloidosis.

In certain embodiments, the method comprises identifying an animal having leptomeningeal amyloidosis and administering to the animal having a the leptomeningeal amyloidosis a therapeutically effective amount of a compound comprising a modified oligonucleotide consisting of 12 to 30 linked nucleosides, wherein the modified oligonucleotide is complementary to human transthyretin.

In certain embodiments the administration is parenteral.

In certain embodiments the administration is intracranial.

In certain embodiments, the intracranial administration is intracerebral administration, intrathecal administration, intraventricular administration, ventricular administration, intracerebroventricular administration, cerebral intraventricular administration or cerebral ventricular administration.

In certain embodiments, the method comprises identifying an animal having a central nervous system related disease or disorder and administering to the animal having central nervous system related disease or disorder by cerebral intraventricular administration a therapeutically effective amount of a compound comprising a modified oligonucleotide consisting of 12 to 30 linked nucleosides, wherein the modified oligonucleotide is complementary to human tranthyretin.

In certain embodiments, the method comprises identifying an animal having a transthyretin amyloid diesease and administering to the animal having transthyretin amyloid diesease by cerebral intraventricular administration a therapeutically effective amount of a compound comprising a modified oligonucleotide consisting of 12 to 30 linked nucleosides, wherein the modified oligonucleotide is complementary to human tranthyretin.

In certain embodiments, transthyretin amyloid diesease, includes by is not, familial amyloid polyneuropathy, senile systemic amyloidosis, or leptomeningeal amyloidosis.

In certain embodiments, the method comprises identifying an animal having leptomeningeal amyloidosis and administering to the animal having a the leptomeningeal amyloidosis by cerebral intraventricular administration a therapeutically effective amount of a compound comprising a modified oligonucleotide consisting of 12 to 30 linked nucleosides, wherein the modified oligonucleotide is complementary to human transthyretin.

In certain embodiments the method results in increased lifespan.

In certain embodiments, the method results in an increased lifespan of days. In certain embodiments, the method results in an increased lifespan of weeks. In certain embodiments, the method results in an increased lifespan of years. In certain embodiments, the method results in an increased lifespan of decades.

### Antisense Compounds

Antisense compounds include, but are not limited to, oligomeric compounds, oligonucleotides, oligonucleosides, oligonucleotide analogs, oligonucleotide mimetics, antisense oligonucleotides, and siRNAs. Antisense compounds may target a nucleic acid, meaning that the antisense compound is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain embodiments an antisense oligonucleotide has a nucleobase sequence that, when written in the 5′ to 3′ direction, comprises the reverse complement of the target segment of a target nucleic acid to which it is targeted.

In certain embodiments an antisense compound targeted to a transthyretin nucleic acid is 12 to 30 subunits in length. In other words, antisense compounds are from 12 to 30 linked subunits. In certain embodiments, the antisense compound is 8 to 80, 12 to 50, 15 to 30, 18 to 24, 19 to 22, or 20 linked subunits. In certain embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In certain embodiments, the linked subunits are linked nucleobases, nucleosides, or nucleotides. In certain embodiments, the antisense compound is an antisense oligonucleotide, and the linked subunits are nucleotides.

In certain embodiments, a shortened or truncated antisense compound targeted to a transthyretin nucleic acid has a single subunit deleted from the 5′ end (5′ truncation), or alternatively from the 3′ end (3′ truncation). A shortened or truncated antisense compound targeted to a transthyretin nucleic acid may have two subunits deleted from the 5′ end, or alternatively may have two subunits deleted from the 3′ end, of the antisense compound. Alternatively, the deleted subunits may be dispersed throughout the antisense compound, for example, in an antisense compound having one subunit deleted from the 5′ end and one subunit deleted from the 3′ end. In certain embodiments, the subunits are nucleobases, nucleosides, or nucleotides.

When a single additional subunit is present in a lengthened antisense compound, the additional subunit may be located at the 5′ or 3′ end of the antisense compound. When two are more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end. In certain embodiments, the subunits are nucleobases, nucleosides, or nucleotides.

It is possible to increase or decrease the length of an antisense compound, such as an antisense oligonucleotide, or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a transthyretin nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleosides that supports RNaseH cleavage is positioned between external regions having a plurality of nucleosides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may, in certain embodiments, include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH2)n—O-2′ bridge, where n=1 or n=2). In certain embodiments, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. Any of the antisense compounds described herein can have a gapmer motif. In certain embodiments, X and Z are the same, in certain other embodiments, they are different. In certain embodiments, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers of the present invention include, but are not limited to, for example 5-10-5, 4-8-4, 4-12-3, 4-12-4, 3-14-3, 2-16-2, 1-18-1, 3-10-3, 2-10-2, 1-10-1 or 2-8-2.

In certain embodiments, the antisense compound as a “wingmer” motif, having a wing-gap or gap-wing configuration, i.e. an X-Y or Y-Z configuration as described above for the gapmer configuration. Thus, wingmer configurations of the present invention include, but are not limited to, for example 5-10, 8-4, 4-12, 12-4, 3-14, 16-2, 18-1, 10-3, 2-10, 1-10 or 8-2.

In certain embodiments, antisense compounds targeted to a transthyretin nucleic acid possess a 5-10-5 gapmer motif.

In certain embodiments, an antisense compound targeted to a transthyretin nucleic acid has a gap-widened motif. In other embodiments, an antisense oligonucleotide targeted to a transthyretin nucleic acid has a gap-widened motif.

In certain embodiments, a gap-widened antisense oligonucleotide targeted to a transthyretin nucleic acid has a gap segment of fourteen 2′-deoxyribonucleotides positioned between wing segments of three chemically modified nucleosides. In certain embodiments, the chemical modification comprises a 2′-sugar modification. In certain embodiments, the chemical modification comprises a 2′-MOE sugar modification.

### Target Nucleic Acids, Target Regions and Nucleotide Sequences

Nucleotide sequences that encode transthyretin include, without limitation, the following: Nucleotide sequences that encode transthyretin include, without limitation, the following:

GENBANK Accession No. BCO20791.1, and incorporated herein as SEQ ID NO: 1 and with GENBANK Accession No NT_{—}010966.10, and incorporated herein as SEQ ID NO: 2.

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the nucleic acid. For example, a target region may encompass a 3′ UTR, a 5′ UTR, an exon, an intron, a coding region, a translation initiation region, translation termination region, or other defined nucleic acid region. The structurally defined regions for transthyretin can be obtained by accession number from sequence databases such as NCBI and such information is incorporated herein by reference. In certain other embodiments, a target region may encompass the sequence from a 5′ target site of one target segment within the target region to a 3′ target site of another target segment within the target region.

Targeting includes determination of at least one target segment to which an antisense compound hybridizes, such that a desired effect occurs. In certain embodiments, the desired effect is a reduction in mRNA target nucleic acid levels. In certain other embodiments, the desired effect is reduction of levels of protein encoded by the target nucleic acid or a phenotypic change associated with the target nucleic acid. In certain embodiments, the reduction is 70% or greater, 75% or greater, 80% or greater, 85% or greater, 90% or greater, 95% or greater, or 100% at a concentration of 100 nM in cells.

A target region may contain one or more target segments. Multiple target segments within a target region may be overlapping. Alternatively, they may be non-overlapping. In certain embodiments, target segments within a target region are separated by no more than about 300 nucleotides. In other embodiments, target segments within a target region are separated by no more than about, 250, 200, 150, 100, 90, 80, 70, 60, 50, 40, 30, 20, or 10 nucleotides on the target nucleic acid. In certain embodiments, target segments within a target region are separated by no more than about 5 nucleotides on the target nucleic acid. In certain embodiments, target segments are contiguous.

Suitable target segments may be found within a 5′ UTR, a coding region, a 3′ UTR, an intron, or an exon. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in transthyretin mRNA levels are indicative of inhibition of transthyretin expression. Reductions in levels of a transthyretin protein are also indicative of inhibition of target mRNA expression. Further, phenotypic changes are indicative of inhibition of transthyretin expression. For example, phenotypic changes may include reduction in amyloid fibril formation and increase in lifespan.

The oligomeric antisense compounds may also be targeted to regions of the target nucleobase sequence (e.g., such as those disclosed in Example 1) comprising nucleobases 1-80, 81-160, 161-240, 241-320, 321-400, 401-480, 481-560, 561-640, 641-650, 6-165, 170-388, 401-420, 425-623, or any combination thereof of SEQ ID NO: 1, and nucleobases 596-8011, 596-615, 1520-1539, 1718-1737, 3880-3899, 4039-4058, 6252-6271, 6967-6986, 7192-8011, or any combination thereof of SEQ ID NO: 2.

Oligomeric compounds may also be targeted to at least a 8 nucleobase portion of nucleobases 596-8011, or 596-615, 1520-1539, 1718-1737, 3880-3899, 4039-4058, 6252-6271, 6967-6986, 7192-8011 of SEQ ID NO: 2, or to nucleobases nucleobases 1-80, 81-160, 161-240, 241-320, 321-400, 401-480, 481-560, 561-640, 641-650, 6-165, 170-388, 401-420, 425-623 of SEQ ID NO: 1, or any combination thereof are also suitable embodiments.

### Hybridization

In certain embodiments, hybridization occurs between an antisense compound disclosed herein and a transthyretin nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a transthyretin nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a transthyretin nucleic acid).

Non-complementary nucleobases between an antisense compound and a transthyretin nucleic acid may be tolerated provided that the antisense compound remains able to specifically hybridize to a target nucleic acid. Moreover, an antisense compound may hybridize over one or more segments of a transthyretin nucleic acid such that intervening or adjacent segments are not involved in the hybridization event (e.g., a loop structure, mismatch or hairpin structure).

In certain embodiments, the antisense compounds provided herein are at least 70%, at least 75%, at least 80%, at least 85%, at least 90%, at least 95%, at least 96%, at least 97%, at least 98% or at least 99% complementary to a transthyretin nucleic acid. Percent complementarity of an antisense compound with a target nucleic acid can be determined using routine methods. For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein are fully complementary (i.e, 100% complementary) to a target nucleic acid. For example, antisense compound may be fully complementary to a transthyretin nucleic acid, or a target region, or a target segment or target sequence thereof. As used herein, “fully complementary” means each nucleobase of an antisense compound is capable of precise base pairing with the corresponding nucleobases of a target nucleic acid.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In certain embodiments, non-complementary nucleobase is located in the wing segment of a gapmer antisense oligonucleotide.

In certain embodiments, antisense compounds up to 20 nucleobases in length comprise no more than 4, no more than 3, no more than 2 or no more than 1 non-complementary nucleobase(s) relative to a target nucleic acid, such as a transthyretin nucleic acid.

In certain embodiments, antisense compounds up to 30 nucleobases in length comprise no more than 6, no more than 5, no more than 4, no more than 3, no more than 2 or no more than 1 non-complementary nucleobase(s) relative to a target nucleic acid, such as a transthyretin nucleic acid.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

In certain embodiments, the antisense compounds provided herein include those comprising a portion which consists of at least 8, 9, 10, 11, 12, 13, 14, 15, 16, 17, 18, 19, or 20 contiguous nucleobases of the nucleobase sequence set forth in SEQ ID NOs: 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, and 133. In certain embodiments, the antisense compounds are complementary to an equal-length portion of SEQ ID NOs: 1 or 2. In certain embodiments, the antisense compounds are at least 75%, 80%, 85%, 90%, 95%, or 100% (fully) complementary to SEQ ID NOs: 1 or 2.

### Identity

The antisense compounds provided herein may also have a defined percent identity to a particular nucleotide sequence, SEQ ID NO, or compound represented by a specific Isis number. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have Identical base pairing relative to the sequence to which it is being compared.

In certain embodiments, the antisense compounds are at least 70%, 75%, 80%, 85%, 90%, 95%, 96%, 97%, 98%, 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a transthyretin nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleotides having modified sugar moieties. Sugar modifications may impart nuclease stability, binding affinity or some other beneficial biological property to the antisense compounds. The furanosyl sugar ring of a nucleoside can be modified in a number of ways including, but not limited to: addition of a substituent group, particularly at the 2′ position; bridging of two non-geminal ring atoms to form a bicyclic nucleic acid (BNA); and substitution of an atom or group such as —S—, —N(R)— or —C(R₁)(R₂) for the ring oxygen at the 4′-position. Modified sugars include, but are not limited to: substituted sugars, especially 2′-substituted sugars having a 2′-F, 2′-OCH₂ (2′-OMe) or a 2′-O(CH₂)₂—OCH₃ (2′-O-methoxyethyl or 2′-MOE) substituent group; and bicyclic modified sugars (BNAs), having a 4′-(CH₂)ₙ—O-2′ bridge, where n=1 or n=22, including α-L-Methyleneoxy (4′-CH2-O-2′) BNA, β-D-Methyleneoxy (4′-CH2-O-2′) BNA and Ethyleneoxy (4′-(CH2)2-O-2′) BNA. Bicyclic modified sugars also include (6′S)-6′ methyl BNA, Aminooxy (4′-CH2-O—N(R)-2′) BNA, Oxyamino (4′-CH2-N(R)—O-2′) BNA wherein, R is, independently, H, a protecting group, or C1-C12 alkyl. The substituent at the 2′ position can also be selected from alyl, amino, azido, thio, O-allyl, O—C1-C10 alkyl, OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl. Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is preferably a methylene (—C₂—)ₙ group bridging the 2′ oxygen atom and the 4′ carbon atom wherein n is 1 or 2. LNAs and preparation thereof are described in WO 98/39352 and WO 99/14226.

In certain embodiments, antisense compounds targeted to a transthyretin nucleic acid comprise one or more nucleotides having modified sugar moieties. In certain embodiments, the modified sugar moiety is 2′-MOE. In certain embodiments, the 2′-MOE modified nucleotides are arranged in a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to a transthyretin nucleic acid comprise one or more modified nucleobases. In certain embodiments, gap-widened antisense oligonucleotides targeted to a transthyretin nucleic acid comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compound targeted to a transthyretin nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in certain embodiments, employed in the methods described herein is a pharmaceutical composition comprising an antisense compound targeted to a transthyretin nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

In certain embodiments, it is beneficial to deliver an antisense oligonucleotide targeted to tranthyretin to the central nervous system (CNS) of an individual suffering from a central nervous system related disorder or transthyretin amyloid diesase. Because the blood-brain barrier is generally impermeable to antisense oligonucleotides administered systemically, antisense oligonucleotides may be delivered to the tissues of the CNS. In certain embodiments, administration of antisense oligonucleotides is directly into the cerebrospinal fluid (CSF). In certain embodiments, delivery to the CSF is achieved by intracrainal administration, intracerebral administration, intrathecal administration, intracerebroventricular administration, cerebral intraventricular administration and cerebral ventricular administration.

Intracranial administration, e.g. intracerebral administration, intrathecal administration, intraventricular administration, ventricular administration, intracerebroventricular administration, cerebral intraventricular administration or cerebral ventricular administration may be achieved through the use of surgically implanted pumps that infuse a therapeutic agent, such as an antisense oligonucleotide, into the CSF. In certain embodiments, an infusion pump may be used. In certain embodiments, the antisense oligonucleotide is continuously infused into the CSF for the entire course of treatment. In certain embodiments, antisense oligonucleotide are delivered to the CSF with an infusion pump such as Medtronic SyncroMed® II pump. The SyncroMed® II pump is surgically implanted according the procedures set forth by the manufacturer. The pump contains a resevoir for retaining one or more a drug solutions, which are pumped at a programmed dose into a catheter that is surgically implanted.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of transthyretin nucleic acids can be tested in vitro in a variety of cell types. Cell types used for such analyses are available from commerical vendors (e.g. American Type Culture Collection, Manassus, Va.; Zen-Bio, Inc., Research Triangle Park, N.C.; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, Hep3B cells and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE® (Invitrogen, Carlsbad, Calif.). Antisense oligonucleotide is mixed with LIPOFECTAMINE® in OPTI-MEM® 1 reduced serum medium (Invitrogen, Carlsbad, Calif.) to achieve the desired concentration of antisense oligonucleotide and a LIPOFECTAMINE® concentration that typically ranges 2 to 12 μg/μL per 100 nM antisense oligonucleotide.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 500 nM.

### RNA Isolation

RNA analysis can be performed on total cellular RNA or poly(A)+ mRNA. Methods of RNA isolation are well known in the art. RNA is prepared using methods well known in the art, for example, using the TRIZOL® Reagent (Invitrogen, Carlsbad, Calif.) according to the manufacturer's recommended protocols.

### Analysis of Inhibition of Target Levels or Expression

Inhibition of levels or expression of a transthyretin nucleic acid can be assayed in a variety of ways known in the art. For example, target nucleic acid levels can be quantitated by, e.g., Northern blot analysis, competitive polymerase chain reaction (PCR), or quantitaive real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISMS 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT, real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN® (Invitrogen, Inc. Carlsbad, Calif.). Cyclophilin A expression is quantified by real time PCR, by being run simultaneously with the target, multiplexing, or separately. Total RNA is quantified using RIBOGREEN® RNA quantification reagent (Invetrogen, Inc. Eugene, Oreg.). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a transthyretin nucleic acid. Methods for designing real-time PCR probes and primers are well known in the art, and may include the use of software such as PRIMER EXPRESS® Software (Applied Biosystems, Foster City, Calif.).

### Analysis of Protein Levels

Antisense inhibition of transthyretin nucleic acids can be assessed by measuring transthyretin protein levels. Protein levels of transthyretin can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, histone deacytelase activity), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of transthyretin and produce phenotypic changes, such as reduction in amyloid fibril formation and increase in lifespan. Amyloid fibril formation may be measured by light scattering and Congo red-binding assay, for example. Lifespan may be measured by increased length of life of a treated animal in comparison to a non-treated animal.

Testing may be performed in normal animals, or in experimental disease models. For administration to animals, antisense oligonucleotides are formulated in a pharmaceutically acceptable diluent, such as phosphate-buffered saline. Administration includes parenteral routes of administration, for example, intravenous, intraarterial, subcutaneous, intraperitoneal, intramuscular injection or infusion, or intracranial e.g; intracerebral administration, intrathecal administration, intraventricular administration, ventricular administration, intracerebroventricular administration, cerebral intraventricular administration or cerebral ventricular administration. Following a period of treatment with antisense oligonucleotides, RNA is isolated from a relevant tissue (e.g., liver tissue for systemic delivery and brain tissue for CNS delivery) and changes in transthyretin nucleic acid expression are measured.

Kits, Research Reagents, Diagnostics, and Therapeutics

The antisense compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The antisense compounds of the invention are useful for research and diagnostics, because these compounds hybridize to nucleic acids encoding transthyretin. For example, oligonucleotides that are shown to hybridize with such efficiency and under such conditions as disclosed herein as to be effective transthyretin inhibitors will also be effective primers or probes under conditions favoring gene amplification or detection, respectively. These primers and probes are useful in methods requiring the specific detection of nucleic acid molecules encoding transthyretin and in the amplification of said nucleic acid molecules for detection or for use in further studies of transthyretin. Hybridization of the antisense oligonucleotides, particularly the primers and probes, of the invention with a nucleic acid encoding transthyretin can be detected by means known in the art. Such means may include conjugation of an enzyme to the oligonucleotide, radiolabelling of the oligonucleotide or any other suitable detection means. Kits using such detection means for detecting the level of transthyretin in a sample may also be prepared.

The specificity and sensitivity of antisense is also harnessed by those of skill in the art for therapeutic uses. Antisense compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compounds can be useful therapeutic modalities that can be configured to be useful in treatment regimes for the treatment of cells, tissues and animals, especially humans.

For therapeutics, an animal, preferably a human, suspected of having a disease or disorder which can be treated by modulating the expression of transthyretin is treated by administering antisense compounds in accordance with this invention. For example, in one non-limiting embodiment, the methods comprise the step of administering to the animal in need of treatment, a therapeutically effective amount of a transthyretin inhibitor. The transthyretin inhibitors of the present invention effectively inhibit the activity of the transthyretin protein or inhibit the expression of the transthyretin protein. In one embodiment, the activity or expression of transthyretin in an animal is inhibited by about 10%. Preferably, the activity or expression of transthyretin in an animal is inhibited by about 30%. More preferably, the activity or expression of transthyretin in an animal is inhibited by 50% or more. Thus, the oligomeric antisense compounds modulate expression of transthyretin mRNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

For example, the reduction of the expression of transthyretin may be measured in serum, adipose tissue, liver or any other body fluid, tissue or organ of the animal. Preferably, the cells contained within said fluids, tissues or organs being analyzed contain a nucleic acid molecule encoding transthyretin protein and/or the transthyretin protein itself.

The antisense compounds of the invention can be utilized in pharmaceutical compositions by adding an effective amount of a compound to a suitable pharmaceutically acceptable diluent or carrier. Use of the compounds and methods of the invention may also be useful prophylactically.

### Certain Combination Therapies

In certain embodiments, one or more pharmaceutical compositions of the present invention are co-administered with one or more other pharmaceutical agents. In certain embodiments, such one or more other pharmaceutical agents are designed to treat the same disease or condition as the one or more pharmaceutical compositions of the present invention. In certain embodiments, such one or more other pharmaceutical agents are designed to treat a different disease or condition as the one or more pharmaceutical compositions of the present invention. In certain embodiments, such one or more other pharmaceutical agents are designed to treat an undesired effect of one or more pharmaceutical compositions of the present invention. In certain embodiments, one or more pharmaceutical compositions of the present invention are co-administered with another pharmaceutical agent to treat an undesired effect of that other pharmaceutical agent. In certain embodiments, one or more pharmaceutical compositions of the present invention and one or more other pharmaceutical agents are administered at the same time. In certain embodiments, one or more pharmaceutical compositions of the present invention and one or more other pharmaceutical agents are administered at different times. In certain embodiments, one or more pharmaceutical compositions of the present invention and one or more other pharmaceutical agents are prepared together in a single formulation. In certain embodiments, one or more pharmaceutical compositions of the present invention and one or more other pharmaceutical agents are prepared separately.

In certain embodiments, pharmaceutical agents that may be co-administered with a pharmaceutical composition of the present invention include analgesics, such as, paracetamol (acetaminophen); non-steroidal anti-inflammatory drugs (NSAIDs), such as, salicylates; narcotic drugs, such as, morphine, and synthetic drugs with narcotic properties such as tramadol.

In certain embodiments, pharmaceutical agents that may be co-administered with a pharmaceutical composition of the present invention include muscle relaxants, such as, benzodiapines and methocarbamol.

In certain embodiments, the second compound is administered prior to administration of a pharmaceutical composition of the present invention. In certain embodiments, the second compound is administered following administration of a pharmaceutical composition of the present invention. In certain embodiments, the second compound is administered at the same time as a pharmaceutical composition of the present invention. In certain embodiments, the dose of a co-administered second compound is the same as the dose that would be administered if the second compound was administered alone. In certain embodiments, the dose of a co-administered second compound is lower than the dose that would be administered if the second compound was administered alone. In certain embodiments, the dose of a co-administered second compound is greater than the dose that would be administered if the second compound was administered alone.

In certain embodiments, the co-administration of a second compound enhances the effect of a first compound, such that co-administration of the compounds results in an effect that is greater than the effect of administering the first compound alone. In certain embodiments, the co-administration results in effects that are additive of the effects of the compounds when administered alone. In certain embodiments, the co-administration results in effects that are supra-additive of the effects of the compounds when administered alone. In certain embodiments, the first compound is an antisense compound. In certain embodiments, the second compound is an antisense compound.

### Formulations

The compounds of the invention may also be admixed, conjugated or otherwise associated with other molecules, molecule structures or mixtures of compounds, as for example, liposomes, receptor-targeted molecules, or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal, including a human, is capable of providing (directly or indirectly) the biologically active metabolite or residue thereof.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety. Sodium salts have been shown to be suitable forms of oligonucleotide drugs.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be parenteral. Parenteral administration includes intravenous, intraarterial, subcutaneous, intraperitoneal or intramuscular injection or infusion; or intracranial, e.g., intracerebral administration, intrathecal administration, intraventricular administration, ventricular administration, intracerebroventricular administration, cerebral intraventricular administration or cerebral ventricular administration. Administration intraventricularly, is preferred to target transthyretin expression in the choroid plexus. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bi layer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Liposomes and their uses are further described in U.S. Pat. No. 6,287,860, which is incorporated herein in its entirety.

The pharmaceutical formulations and compositions of the present invention may also include surfactants. Surfactants and their uses are further described in U.S. Pat. No. 6,287,860, which is incorporated herein in its entirety.

In one embodiment, the present invention employs various penetration enhancers to affect the efficient delivery of nucleic acids, particularly oligonucleotides. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

Compositions and formulations for parenteral administration, including intravenous, intraarterial, subcutaneous, intraperitoneal, intramuscular injection or infusion, or intracranial may include sterile aqueous solutions which may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more oligomeric compounds and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of antisense compounds and other non-antisense drugs are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Alternatively, compositions of the invention may contain two or more antisense compounds targeted to different regions of the same nucleic acid target. Numerous examples of antisense compounds are known in the art. Two or more combined compounds may be used together or sequentially.

### Dosing

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual oligonucleotides, and can generally be estimated based on EC₅₀s found to be effective in in vitro and in vivo animal models. In general, dosage is from 0.01 μg to 100 g per kg of body weight, and may be given once or more daily, weekly, monthly or yearly, or at desired intervals. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily.

While the present invention has been described with specificity in accordance with certain of its preferred embodiments, the following examples serve only to illustrate the invention and are not intended to limit the same. Each of the references, GenBank accession numbers, and the like recited in the present application is incorporated herein by reference in its entirety.

### Related Disclosures

US 20050244869 is commonly owned with the instant application and is incorporated herein by reference in its entirety.

### EXAMPLES

### Example 1

### Antisense Inhibition of Human Transthyretin Expression by Chimeric Phosphorothioate Oligonucleotides Having 2′-MOE Wings and a Deoxy Gap

In accordance with the present invention, a series of antisense compounds was designed to target different regions of the human transthyretin RNA, using published sequences (GenBank accession number BCO20791.1, incorporated herein as SEQ ID NO: 1, and nucleotides 2009236 to 2017289 of the sequence with GenBank accession number NT_{—}010966.10, incorporated herein as SEQ ID NO: 2). The compounds are shown in Table 1. “Target site” indicates the first (5′-most) nucleotide number on the particular target sequence to which the compound binds. All compounds in Table 1 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-(2-methoxyethyl) nucleotides, also known as 2′-MOE nucleotides. The internucleoside (backbone) linkages are phosphorothioate (P=5) throughout the oligonucleotide. All cytidine residues are 5-methylcytidines. The compounds were analyzed for their effect on human transthyretin mRNA levels by quantitative real-time PCR as described in other examples herein. Data are averages from two experiments in which HepG2 cells were treated with 50 nM of the antisense oligonucleotides of the present invention. The positive control ISIS 18078 (GTGCGCGCGAGCCCGAAATC, SEQ ID NO: 9) was used for this assay. If present, “N.D.” indicates “no data”.

TABLE 1
Inhibition of human transthyretin mRNA levels by chimeric phosphorothioate
oligonucleotides having 2′-MOE wings and a deoxy gap

| | | TARGET | | | | SEQ |
|---|---|---|---|---|---|---|
| | | SEQ | TARGET | | % | ID |
| ISIS # | REGION | ID NO | SITE | SEQUENCE | INHIB | NO |
| 304237 | Exon 1: | 11 | 596 | aaacactcaccgtagggcca | 6 | 12 |
| | Intron 1 | | | | | |
| | junction | | | | | |
| 304238 | Intron 1: | 11 | 1520 | caccggtgccctgggtgtag | 0 | 13 |
| | Exon 2 | | | | | |
| | junction | | | | | |
| 304239 | Intron 2 | 11 | 1718 | tgagcctctctctaccaagt | 0 | 14 |
| 304240 | Exon 3: | 11 | 3880 | gtatactcacctctgcatgc | 33 | 15 |
| | Intron 3 | | | | | |
| | junction | | | | | |
| 304241 | Intron 3 | 11 | 4039 | ttctcagagtgttgtgaatt | 0 | 16 |
| 304242 | Intron 3 | 11 | 6252 | actctgcataaatacatttt | 0 | 17 |
| 304243 | Intron 3 | 11 | 6967 | tcttgttttgcaaattcacg | 0 | 18 |
| 304244 | Intron 3 | 11 | 7192 | tgaataccacctatgagaga | 0 | 19 |
| 304245 | 5′UTR | 4 | 6 | ctgccaagaatgagtggact | 33 | 20 |
| 304246 | Start Codon | 4 | 18 | tgagaagccatcctgccaag | 6 | 21 |
| 304247 | Start Codon | 4 | 25 | cagacgatgagaagccatcc | 2 | 22 |
| 304248 | Coding | 4 | 30 | aggagcagacgatgagaagc | 10 | 23 |
| 304249 | Coding | 4 | 59 | acacaaataccagtccagca | 33 | 24 |
| 304250 | Coding | 4 | 60 | gacacaaataccagtccagc | 0 | 25 |
| 304251 | Coding | 4 | 66 | gcctcagacacaaataccag | 14 | 26 |
| 304252 | Coding | 4 | 75 | gtagggccagcctcagacac | 3 | 27 |
| 304253 | Coding | 4 | 86 | caccggtgcccgtagggcca | 16 | 28 |
| 304254 | Coding | 4 | 91 | ggattcaccggtgcccgtag | 32 | 29 |
| 304255 | Coding | 4 | 100 | aggacacttggattcaccgg | 47 | 30 |
| 304256 | Coding | 4 | 105 | atcagaggacacttggattc | 0 | 31 |
| 304257 | Coding | 4 | 110 | tgaccatcagaggacacttg | 21 | 32 |
| 304258 | Coding | 4 | 114 | actttgaccatcagaggaca | 16 | 33 |
| 304259 | Coding | 4 | 126 | acagcatctagaactttgac | 33 | 34 |
| 304260 | Coding | 4 | 133 | gcctcggacagcatctagaa | 34 | 35 |
| 304261 | Coding | 4 | 146 | tgatggcaggactgcctcgg | 16 | 36 |
| 304262 | Coding | 4 | 170 | ttctgaacacatgcacggcc | 41 | 37 |
| 304263 | Coding | 4 | 185 | tgtcatcagcagcctttctg | 8 | 38 |
| 304264 | Coding | 4 | 197 | atggctcccaggtgtcatca | 34 | 39 |
| 304265 | Coding | 4 | 203 | aggcaaatggctcccaggtg | 15 | 40 |
| 304266 | Coding | 4 | 210 | ttcccagaggcaaatggctc | 0 | 41 |
| 304267 | Coding | 4 | 217 | actggttttcccagaggcaa | 56 | 42 |
| 304268 | Coding | 4 | 222 | gactcactggttttcccaga | 0 | 43 |
| 304269 | Coding | 4 | 232 | cagctctccagactcactgg | 44 | 44 |
| 304270 | Coding | 4 | 239 | gcccatgcagctctccagac | 14 | 45 |
| 304271 | Coding | 4 | 244 | tgtgagcccatgcagctctc | 3 | 46 |
| 304272 | Coding | 4 | 250 | ctcagttgtgagcccatgca | 36 | 47 |
| 304273 | Coding | 4 | 257 | attcctcctcagttgtgagc | 10 | 48 |
| 304274 | Coding | 4 | 264 | tctacaaattcctcctcagt | 34 | 49 |
| 304275 | Coding | 4 | 278 | ctttgtatatcccttctaca | 43 | 50 |
| 304276 | Coding | 4 | 298 | agatttggtgtctatttcca | 1 | 51 |
| 304277 | Coding | 4 | 314 | caagtgccttccagtaagat | 14 | 52 |
| 304278 | Coding | 4 | 323 | gggagatgccaagtgccttc | 53 | 53 |
| 304279 | Coding | 4 | 342 | tctgcatgctcatggaatgg | 42 | 54 |
| 304280 | Coding | 4 | 353 | tgaataccacctctgcatgc | 7 | 55 |
| 304281 | Coding | 4 | 360 | ttggctgtgaataccacctc | 5 | 56 |
| 304282 | Coding | 4 | 369 | ccggagtcgttggctgtgaa | 16 | 57 |
| 304283 | Coding | 4 | 401 | tcagcagggcggcaatggtg | 1 | 58 |
| 304284 | Coding | 4 | 425 | ccgtggtggaataggagtag | 63 | 59 |
| 304285 | Coding | 4 | 427 | agccgtggtggaataggagt | 53 | 60 |
| 304286 | Coding | 4 | 431 | cgacagccgtggtggaatag | 56 | 61 |
| 304287 | Coding | 4 | 438 | ttggtgacgacagccgtggt | 92 | 62 |
| 304288 | Coding | 4 | 440 | gattggtgacgacagccgtg | 70 | 63 |
| 304289 | Coding | 4 | 442 | gggattggtgacgacagccg | 73 | 64 |
| 304290 | Coding | 4 | 443 | tgggattggtgacgacagcc | 83 | 65 |
| 304291 | Coding | 4 | 449 | attccttgggattggtgacg | 45 | 66 |
| 304292 | Stop Codon | 4 | 450 | cattccttgggattggtgac | 27 | 67 |
| 304293 | Stop Codon | 4 | 451 | tcattccttgggattggtga | 20 | 68 |
| 304294 | Stop Codon | 4 | 460 | agaagtccctcattccttgg | 37 | 69 |
| 304295 | 3′UTR | 4 | 472 | gtccactggaggagaagtcc | 47 | 70 |
| 304296 | 3′UTR | 4 | 481 | gtccttcaggtccactggag | 86 | 71 |
| 304297 | 3′UTR | 4 | 489 | catccctcgtccttcaggtc | 76 | 72 |
| 304298 | 3′UTR | 4 | 501 | tacatgaaatcccatccctc | 52 | 73 |
| 304299 | 3′UTR | 4 | 507 | cttggttacatgaaatccca | 78 | 74 |
| 304300 | 3′UTR | 4 | 513 | aatactcttggttacatgaa | 52 | 75 |
| 304301 | 3′UTR | 4 | 526 | ttagtaaaaatggaatactc | 20 | 76 |
| 304302 | 3′UTR | 4 | 532 | actgctttagtaaaaatgga | 57 | 77 |
| 304303 | 3′UTR | 4 | 539 | tgaaaacactgctttagtaa | 54 | 78 |
| 304304 | 3′UTR | 4 | 546 | tatgaggtgaaaacactgct | 48 | 79 |
| 304305 | 3′UTR | 4 | 551 | tagcatatgaggtgaaaaca | 68 | 80 |
| 304306 | 3′UTR | 4 | 559 | ttctaacatagcatatgagg | 72 | 81 |
| 304307 | 3′UTR | 4 | 564 | tggacttctaacatagcata | 79 | 82 |
| 304308 | 3′UTR | 4 | 572 | tctctgcctggacttctaac | 75 | 83 |
| 304309 | 3′UTR | 4 | 578 | ttattgtctctgcctggact | 83 | 84 |
| 304310 | 3′UTR | 4 | 595 | cctttcacaggaatgtttta | 46 | 85 |
| 304311 | 3′UTR | 4 | 597 | tgcctttcacaggaatgttt | 79 | 86 |
| 304312 | 3′UTR | 4 | 598 | gtgcctttcacaggaatgtt | 80 | 87 |
| 304313 | 3′UTR | 4 | 600 | aagtgcctttcacaggaatg | 68 | 88 |
| 304314 | 3′UTR | 4 | 604 | tgaaaagtgcctttcacagg | 8 | 89 |

As shown in Table 1, SEQ ID NOs 15, 20, 24, 29, 30, 34, 35, 37, 39, 42, 44, 47, 49, 50, 53, 54, 59, 60, 61, 62, 63, 64, 65, 66, 67, 69, 70, 71, 72, 73, 74, 75, 77, 78, 79, 80, 81, 82, 83, 84, 85, 86, 87 and 88 demonstrated at least 27% inhibition of human transthyretin expression in this assay and are therefore preferred. More preferred are SEQ ID NOs 84, 87, and 86. The target regions to which these preferred sequences are complementary are herein referred to as “preferred target segments” and are therefore preferred for targeting by compounds of the present invention. These preferred target segments are shown in Table 2. These sequences are shown to contain thymine (T) but one of skill in the art will appreciate that thymine (T) is generally replaced by uracil (U) in RNA sequences. The sequences represent the reverse complement of the preferred antisense compounds shown in Table 1. “Target site” indicates the first (5′-most) nucleotide number on the particular target nucleic acid to which the oligonucleotide binds. Also shown in Table 2 is the species in which each of the preferred target segments was found.

TABLE 2
Sequence and position of preferred target segments identified in transthyretin.

| | | | | REV | | |
|---|---|---|---|---|---|---|
| | TARGET | | | COMP | | SEQ |
| SITE | SEQ ID | TARGET | | OF SEQ | | ID |
| ID | NO | SITE | SEQUENCE | ID | ACTIVE IN | NO |
| 220029 | 11 | 3880 | gcatgcagaggtgagtatac | 15 | H. sapiens | 90 |
| 220034 | 4 | 6 | agtccactcattcttggcag | 20 | H. sapiens | 91 |
| 220038 | 4 | 59 | tgctggactggtatttgtgt | 24 | H. sapiens | 92 |
| 220043 | 4 | 91 | ctacgggcaccggtgaatcc | 29 | H. sapiens | 93 |
| 220044 | 4 | 100 | ccggtgaatccaagtgtcct | 30 | H. sapiens | 94 |
| 220048 | 4 | 126 | gtcaaagttctagatgctgt | 34 | H. sapiens | 95 |
| 220049 | 4 | 133 | ttctagatgctgtccgaggc | 35 | H. sapiens | 96 |
| 220051 | 4 | 170 | ggccgtgcatgtgttcagaa | 37 | H. sapiens | 97 |
| 220053 | 4 | 197 | tgatgacacctgggagccat | 39 | H. sapiens | 98 |
| 220056 | 4 | 217 | ttgcctctgggaaaaccagt | 42 | H. sapiens | 99 |
| 220058 | 4 | 232 | ccagtgagtctggagagctg | 44 | H. sapiens | 100 |
| 220061 | 4 | 250 | tgcatgggctcacaactgag | 47 | H. sapiens | 101 |
| 220063 | 4 | 264 | actgaggaggaatttgtaga | 49 | H. sapiens | 102 |
| 220064 | 4 | 278 | tgtagaagggatatacaaag | 50 | H. sapiens | 103 |
| 220067 | 4 | 323 | gaaggcacttggcatctccc | 53 | H. sapiens | 104 |
| 220068 | 4 | 342 | ccattccatgagcatgcaga | 54 | H. sapiens | 105 |
| 220073 | 4 | 425 | ctactcctattccaccacgg | 59 | H. sapiens | 106 |
| 220074 | 4 | 427 | actcctattccaccacggct | 60 | H. sapiens | 107 |
| 220075 | 4 | 431 | ctattccaccacggctgtcg | 61 | H. sapiens | 108 |
| 220076 | 4 | 438 | accacggctgtcgtcaccaa | 62 | H. sapiens | 109 |
| 220077 | 4 | 440 | cacggctgtcgtcaccaatc | 63 | H. sapiens | 110 |
| 220078 | 4 | 442 | cggctgtcgtcaccaatccc | 64 | H. sapiens | 111 |
| 220079 | 4 | 443 | ggctgtcgtcaccaatccca | 65 | H. sapiens | 112 |
| 220080 | 4 | 449 | cgtcaccaatcccaaggaat | 66 | H. sapiens | 113 |
| 220081 | 4 | 450 | gtcaccaatcccaaggaatg | 67 | H. sapiens | 114 |
| 220083 | 4 | 460 | ccaaggaatgagggacttct | 69 | H. sapiens | 115 |
| 220084 | 4 | 472 | ggacttctcctccagtggac | 70 | H. sapiens | 116 |
| 220085 | 4 | 481 | ctccagtggacctgaaggac | 71 | H. sapiens | 117 |
| 220086 | 4 | 489 | gacctgaaggacgagggatg | 72 | H. sapiens | 118 |
| 220087 | 4 | 501 | gagggatgggatttcatgta | 73 | H. sapiens | 119 |
| 220088 | 4 | 507 | tgggatttcatgtaaccaag | 74 | H. sapiens | 120 |
| 220089 | 4 | 513 | ttcatgtaaccaagagtatt | 75 | H. sapiens | 121 |
| 220091 | 4 | 532 | tccatttttactaaagcagt | 77 | H. sapiens | 122 |
| 220092 | 4 | 539 | ttactaaagcagtgttttca | 78 | H. sapiens | 123 |
| 220093 | 4 | 546 | agcagtgttttcacctcata | 79 | H. sapiens | 124 |
| 220094 | 4 | 551 | tgttttcacctcatatgcta | 80 | H. sapiens | 125 |
| 220095 | 4 | 559 | cctcatatgctatgttagaa | 81 | H. sapiens | 126 |
| 220096 | 4 | 564 | tatgctatgttagaagtcca | 82 | H. sapiens | 127 |
| 220097 | 4 | 572 | gttagaagtccaggcagaga | 83 | H. sapiens | 128 |
| 220098 | 4 | 578 | agtccaggcagagacaataa | 84 | H. sapiens | 129 |
| 220099 | 4 | 595 | taaaacattcctgtgaaagg | 85 | H. sapiens | 130 |
| 220100 | 4 | 597 | aaacattcctgtgaaaggca | 86 | H. sapiens | 131 |
| 220101 | 4 | 598 | aacattcctgtgaaaggcac | 87 | H. sapiens | 132 |
| 220102 | 4 | 600 | cattcctgtgaaaggcactt | 88 | H. sapiens | 133 |

As these “preferred target segments” have been found by experimentation to be open to, and accessible for, hybridization with the antisense compounds of the present invention, one of skill in the art will recognize or be able to ascertain, using no more than routine experimentation, further embodiments of the invention that encompass other compounds that specifically hybridize to these preferred target segments and consequently inhibit the expression of transthyretin.

According to the present invention, antisense compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 2

### Cerebral Intraventricular Administration of Antisense Oligonucleotides on Transthyretin Expression in the Choroid Plexus

Subcutaneous Administration of Antisense Oligonucleotide:

Two groups of mice (6 per group) were treated subcutaneously with antisense oligonucleotide ISIS 304309, 25 mg/kg, twice a week for two weeks or an equal volume of normal saline. Mice were sacrificed four days after the last injection. Blood was obtained to determine human transthyretin concentration. Brain and liver tissues were divided with ½ frozen for transthyretin mRNA quantification and ½ fixed (ten percent formalin) for immunohistochemistry. Controls and experimental animals were matched for comparable initial weight (average 44 gm) and sex (three males, three females). Antisense oligonucleotide was administered as a 5 mg/ml solution.

Cerebral Intraventricular Administration of Antisense Oligonucleotide

Mice transgenic for human transthyretin Ile84Ser received either saline or antisense oligonucleotide ISIS 304309 following placement of an intraventricular cannula that was connected to a subcutaneously implanted Alzet 2004 osmotic pump. All animals were anesthetized with isoflurane and cannulas were placed in the right lateral ventricle at a depth of 2.5 mm (stereotactic coordinates: 1.6 mm lateral and 0.7 mm posterior to bregma). Postoperatively Ibuprofen was supplied in the drinking water. After 28 days of treatment, mice were sacrificed and brains were divided sagittally; ½ was frozen for mRNA quantification and ½ fixed in ten percent formalin for immunohistochemistry. Livers were frozen for mRNA quantification.

RNA Analyses.

Total RNA was isolated from frozen brains and livers by homogenization in TRIzol reagent (Invitrogen), and reverse transcription reactions were performed using the high-capacity cDNA archive kit (PE Applied Biosystems, Foster City, Calif.) as previously describe. Real-time quantitative PCR was performed using the 5 fluorogenic nuclease assay and an ABI Prism 7900 HT Sequence Detection System (PE Applied Biosystems) to determine the level of human transthyretin mRNA, and samples were normalized by determining the relative abundance of ribosome protein 36B4 mRNA. Primer and probe sequences were as follows: human transthyretin forward primer 5-CCGAGGCAGTCCTGCCATCA-3 (SEQ ID NO: 3); human transthyretin reverse primer 5-GCTCCCAGGTGTCATCAGCA-3 (SEQ ID NO: 4); human transthyretin Taqman probe 5-TGTGGCCGTGCATGTGTTCAGAAAGG-3 (SEQ ID NO: 5); mouse 36B4 forward primer 5-GGCCCGAGAAGACCTCCTT-3 (SEQ ID NO: 6); mouse 36B4 reverse primer 5-TCAATGGTGCCTCTGGAGATT-3 (SEQ ID NO: 7); and mouse 36B4 TaqMan probe 5-CCAGGCTTTGGGCATCACCACG-3 (SEQ ID NO: 8). PCR reactions were run in triplicate reactions containing Universal PCR Master Mix (PE Applied Biosystems), 4 pmol of each forward and reverse primer, 3 pmol of probe, and cDNA. Two-step PCR cycling was carried out as follows: 50° C., 2 minutes for 1 cycle; 95° C., 10 minutes for 1 cycle; and 95° C., 15 seconds and 60° C., 1 minute for 40 cycles.

Immunohistochemistry.

Sections of paraffin embedded, formalin fixed tissues were deparaffinized and rehydrated. Endogenase peroxidase was quenched using 0.3 percent (V/V) hydrogen peroxide in methanol for 30 minutes. Sections were incubated sequentially in 1.5 percent goat serum for 30 minutes, rabbit anti-human transthyretin antiserum 1:1000 (Dako Cytomation, Inc., Carpinteria, Calif.) for one hour, biotinylated goat anti-rabbit immunoglobulin G (1:200) (Vector Laboratories, Burlingame, Calif.) for 30 minutes, ABC reagent (Vector Laboratories) for 45 minutes and substrate for three to seven minutes. Horseradish peroxidase substrate was prepared using FAST diaminobenzadine and urea H₂0₂ tablets (Sigma-Aldrich, St. Louis, Mo.). Tissues were counterstained with hematoxylin. Staining was graded as 0 to 4+ and representative sections photographed on a Nikon Microphot-SA microscope with RT WE SPOT digital camera.

Measurement of Human Transthyretin.

Human transthyretin serum concentrations were determined by nephthalometry (Beckman Assay 360) with standard clinical pathology calibration. Transthyretin levels were expressed as percent of the baseline transthyretin level for each individual animal.

Statistics:

One-way analysis of normalized qPCR log 10 values was performed using JMP5.1 software. Group means were compared using Dunnett's method with control. P-values less than 0.05 were considered statistically significant.

Subcutaneous Administration of Antisense Oligonucleotide.

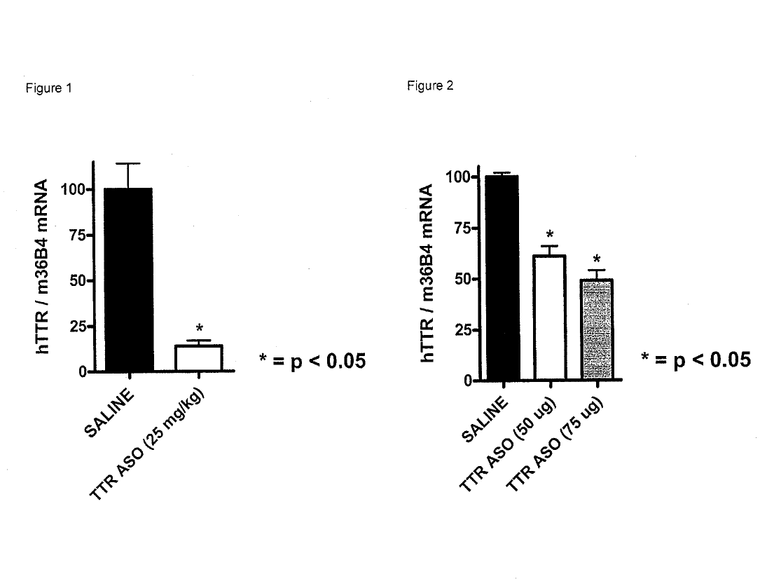

Animals (six) sacrificed four days after last antisense oligonucleotide treatment (25 mg/kg twice weekly for two weeks) had a mean serum human transthyretin concentration of 29.5±4.5 mg/di (21 percent of baseline transthyretin concentration). Animals (six) treated with saline had a mean serum transthyretin concentration of 145.1±25.4 mg/dl (100 percent of baseline transthyretin concentration). Transthyretin mRNA levels in livers of antisense oligonucleotide treated mice were 14±3 percent of the transthyretin mRNA level for saline treated mice (P<0.05) (FIG. 1). Human transthyretin mRNA levels of brains of antisense oligonucleotide treated mice were 78±6 percent of brain levels for saline treated mice (P=0.114).

Cerebral Intraventricular Administration of Antisense Oligonucleotide ISIS 304309.

The effects of transthyretin antisense oligonucleotide treatment on transthyretin levels in brain were examined following cerebral intraventricular administration. Seven mice received 50 μg of antisense oligonucleotide ISIS 304309/day, six received 75 μg/day, and five control mice received saline for 28 days. Levels of human transthyretin mRNA in brain tissues were measured and expressed as percent of the mean level for saline treated mice (FIG. 2). Mean brain human transthyretin mRNA levels for animals receiving 50 μg/day was 61±5 percent of control value (P=0.001); for animals receiving 75 μg/day mean human transthyretin mRNA was 49±5 percent of control value (P=0.00009). Murine brain transthyretin mRNA levels for mice treated with 50 μg/day antisense oligonucleotide did not differ significantly from saline controls (P=0.70), whereas murine brain transthyretin mRNA levels for mice treated with 75 μg/day were moderately suppressed 71±5 percent (P=0.02). Levels of human transthyretin mRNA in liver tissues were not suppressed in intraventricular antisense oligonucleotide treated animals receiving antisense oligonucleotide 50 μg/day (135 percent of control level) or antisense oligonucleotide 75 μg/day (118 percent of control level). Murine transthyretin mRNA levels in liver tissues were slightly lower in 50 μg/day and 75 μg/day animals (88 percent and 81 percent of control respectively).

Immunohistochemistry.

Liver and choroid plexus staining by immunohistochemistry with anti-human transthyretin was arbitrarily graded as 0 to 4+. Degree of staining of liver sections from mice treated with antisense oligonucleotide by subcutaneous administration had a mean of 1.1±0.31; liver tissues from animals that received saline had mean staining 3.8±0.17. Choroid plexus staining for animals treated with antisense oligonucleotide by subcutaneous injection was 3.5±0.35 compared to 3.0±0.37 for saline treated animals.

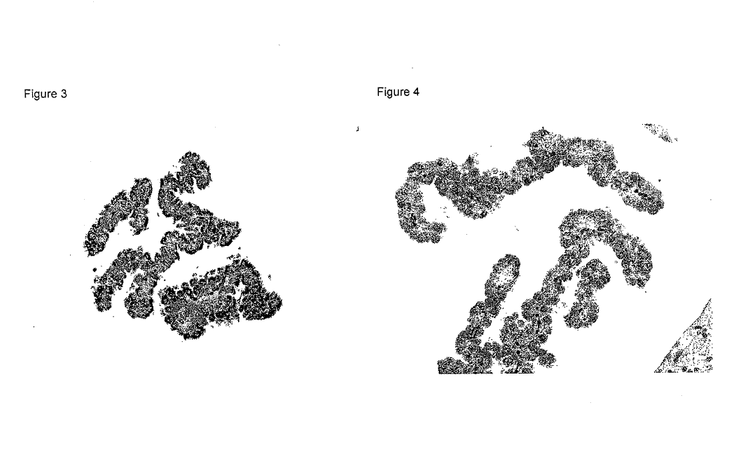

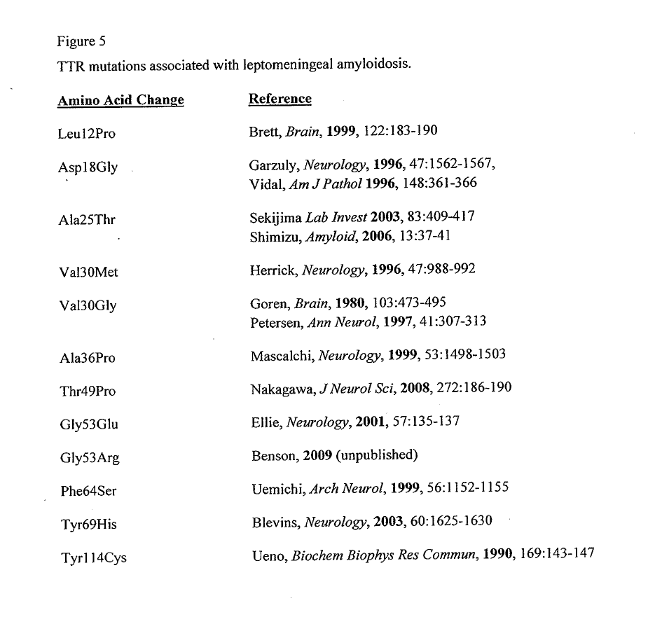

Choroid plexus staining for transthyretin in brain sections from intraventricular antisense oligonucleotide treated animals was less than saline treated animals, 1.3±0.73 for 50 μg/day and 1.5±0.44 for 75 μg/day versus 2.2±0.85 but these values did not reach the P<0.05 level of significance (FIGS. 3 and 4).

Thus, subcutaneous administration of human transthyretin specific antisense oligonucleotide significantly suppressed hepatic transthyretin synthesis but did not give significant suppression of human transthyretin expression by the choroid plexus. Administration of the transthyretin antisense oligonucleotide via the cerebral intraventricular system did significantly suppress choroid expression of transthyretin as measured by transthyretin mRNA levels. Immunohistochemical staining of choroid plexus with anti-human transthyretin was also consistent with suppression of transthyretin synthesis following cerebral intraventricular administration of antisense oligonucleotide, but considerable variability was noted amongst treated animals, perhaps a result of tissue sampling. In addition, immunohistochemistry may not resolve differences in the magnitude of protein expression that in this instance, are likely to be 25-40 percent of normal. Cerebral intraventricular administration of antisense oligonucleotide had no effect on hepatic human transthyretin mRNA levels. Shown herein, local administration of transthyretin antisense oligonucleotide to brain via cerebral intraventricular injection resulted in a dose-dependent reduction in transthyretin levels in brain, which can be used for the treatment of diseases related to the overexpression of transthyretin in the choroid plexus.

All of the applications, patents and references cited are hereby incorporated herein by reference.

It will be apparent to those skilled in the art that various modifications and variations can be made in the methods and compositions of the present invention without departing from the spirit or scope of the invention. Thus it is intended that the present invention cover modifications and variations of this invention.

What is claimed is:

1. A method of inhibiting transthyretin in the brain of an animal comprising;
administering a modified oligonucleotide consisting of 12 to 30 linked nucleosides, wherein the modified oligonucleotide is complementary to human transthyretin; and thereby inhibiting transthyretin in the brain of an animal.

2. The method of claim 1, wherein the transthyretin inhibitor is a nucleic acid.

3. The method of claim 2, wherein the nucleic acid is a modified oligonucleotide.

4. The method of claim 3, wherein said modified oligonucleotide is a single-stranded oligonucleotide.

5. The method of claim 4, wherein the nucleobase sequence of the modified oligonucleotide is 100% complementary to human transthyretin.

6. The method of claim 4, wherein at least one internucleoside linkage is a modified internucleoside linkage.

7. The method of claim 6, wherein each internucleoside linkage is a phosphorothioate internucleoside linkage.

8. The method of claim 4, wherein at least one nucleoside comprises a modified sugar.

9. The method of claim 8, wherein at least one modified sugar is a bicyclic sugar.

10. The method of claim 8, wherein at least one modified sugar comprises a 2′-O-methoxyethyl.

11. The method of claim 4, wherein at least one nucleoside comprises a modified nucleobase.

12. A method comprising identifying an animal having a central nervous system disorder; and
administering to the brain of the animal having a central nervous system disorder a therapeutically effective amount of a transthyretin inhibitor.

13. The method of claim 12, wherein the central nervous system disorder is a transthyretin amyloid disease.

14. The method of claim 13, wherein the transthyretin amyloid disease consists of leptomeningeal amyloidosis or familial amyloid polyneuropathy.

15. A method of reducing amyloid fibril formation in an animal comprising;
administering to the brain of the animal a therapeutically effective amount of a compound comprising a modified oligonucleotide consisting of 12 to 30 linked nucleosides, wherein the modified oligonucleotide is complementary to human transthyretin; and
thereby reducing amyloid fibril formation in an animal.

16. The method of claim 15, wherein the animal has a central nervous system related disorder.

17. The method of claim 16, wherein the central nervous system related disorder is transthyretin amyloid disease.

18. The method of claim 17, wherein the transthyretin amyloid disease consists of leptomeningeal amyloidosis or familial amyloid polyneuropathy.

19. The method of claim 16, wherein the administering results in a 20% reduction of amyloid fibril formation.

20. The method of claim 19, wherein the administering results in a 30% reduction of amyloid fibril formation.

21. The method of claim 20, wherein the administering results in a 40% reduction of amyloid fibril formation.

22. The method of claim 21, wherein the administering results in a 50% reduction of amyloid fibril formation.

23. The method of claim 22, wherein the administering results in a 60% reduction of amyloid fibril formation.

24. The method of claim 23, wherein the administering results in a 70% reduction of amyloid fibril formation.

25. The method of claim 24, wherein the administering results in an 80% reduction of amyloid fibril formation.

26. The method of claim 25, wherein the administering results in a 90% reduction of amyloid fibril formation.

27. The method of claim 26, wherein the administering results in a 100% reduction of amyloid fibril formation.

28. A method of preventing, ameliorating, or treating of a central nervous system disorder in an animal comprising;
identifying an animal having a central nervous system disorder;
administering to the brain of the animal a therapeutically effective amount of a compound comprising a modified oligonucleotide consisting of 12 to 30 linked nucleosides, wherein the modified oligonucleotide is complementary to human transthyretin; and
thereby preventing, ameliorating, or treating of a central nervous system disorder in an animal.

29. The method of claim 28, wherein the central nervous system disorder is a transthyretin amyloid disease.

30. The method of claim 29, wherein the transthyretin amyloid disease consists of leptomeningeal amyloidosis or familial amyloid polyneuropathy.

31. A method of inhibiting transthyretin in the choroid plexus of an animal comprising;
administering a modified oligonucleotide consisting of 12 to 30 linked nucleosides, wherein the modified oligonucleotide is complementary to human transthyretin; and thereby inhibiting transthyretin in the brain of an animal.

32. The method of claims 1, 12, 15, 28, and 31 wherein the administering comprises intracranial administration.

33. The method of claim 32, wherein intracranial administration consists of intracerebral administration, intrathecal administration, intraventricular administration, ventricular administration, intracerebroventricular administration, cerebral intraventricular administration or cerebral ventricular administration.
